(19) United States
(12) Patent Application Publication
(10) Pub. No.: US 2026/0074037 A1
(43) Pub. Date: Mar. 12, 2026

(54) STRATEGIC CONTENT REDUCTION TO FACILITATE COMMUNICATION EFFICIENCY

(71) Applicant: Oracle International Corporation, Redwood Shores, CA (US)

(72) Inventors: Ajit Sutar, Belmont, CA (US); Madhvi Sharma, Seattle, WA (US); Mikhail Mikhailov, Seattle, WA (US); Priyanka Kancharla, New York, NY (US); Alexander Graff, Portland, ME (US); Zachary Brown, Kansas City, MO (US); Arasu Narayan, Auburn, WA (US); Amanda Steffon, Bountiful, UT (US); Katelin Brown, Bountiful, UT (US); Joseph Kaufmann, Austin, TX (US); Kate D'Orazio, Brooklyn, NY (US); Alireza Dibazar, Belmont, CA (US); Suraj Bhat, Seattle, WA (US)

(73) Assignee: Oracle International Corporation, Redwood Shores, CA (US)

(21) Appl. No.: 19/079,655

(22) Filed: Mar. 14, 2025

Related U.S. Application Data
(60) Provisional application No. 63/691,922, filed on Sep. 6, 2024.
(60) Provisional application No. 63/712,216, filed on Oct. 25, 2024.

(51) Int. Cl. G16H 10/60; G06F 9/54; G06F 40/40

(52) U.S. Cl. CPC G16H 10/60; G06F 9/547; G06F 40/40

(57) ABSTRACT

The present disclosure relates to generating a structured representation, particularly a summary, for a given subject by leveraging one or more generative artificial intelligence (GenAI) models based on one or more unstructured portions of electronic health record data of the subject. The techniques, as disclosed herein, may utilize a long record application programmable interface (API) to retrieve and process the one or more unstructured portions of electronic health record data of the subject into a semi-structured format. The disclosed techniques further utilize the one or more GenAI models to generate a prompt based on the semi-structured format, extract relevant data elements based on the prompt, and transform the data elements into a strategically reduced summary. The generated summary may assist users in making quick, informed decisions while reducing the need for exhaustive manual chart reviews of the subject.

### CROSS-REFERENCE OF RELATED APPLICATIONS

This application is related to U.S. Provisional Patent Application No. 63/691,922, filed on Sep. 6, 2024, and U.S. Provisional Patent Application No. 63/712,216, filed on Oct. 25, 2024, which are incorporated by reference in their entireties for all purposes.

### BACKGROUND

In a healthcare environment, care managers may foster long-term relationships with high-risk subjects to improve their health outcomes. Care managers collaborate closely with subjects to develop tailored care plans that focus not only on managing specific health conditions, but also on promoting overall well-being of a subject. Such care plans typically include achievable goals and interventions including dietary changes, exercise regimens or lifestyle modifications that may be aimed at helping subjects better control their health. Additionally, subjects that are enrolled in a care management program may also benefit from receiving timely intervention through their care manager (or care management team) to help mitigate rising risks and avoid emergency department visits or hospital re-admissions by addressing potential health issues before they escalate into more serious conditions. Thus, the role of a care manager is multifaceted, encompassing the coordination of medical treatments, building relationships with subjects, advocating for their health, and connecting them with necessary social and community support systems to ensure continuous care.

Despite the critical nature of their work, care managers face numerous challenges in executing such responsibilities effectively. For instance, care managers may often face overwhelming workloads, excessive documentation and/or administrative burdens. The sheer workload, detailed chart reviews and the time spent cold calling eligible subjects to enroll in care management programs, compounded by challenges e.g., unreachable patients, unreturned voicemails or subjects who decline enrollment, may further strain care managers. Such increased workloads, exacerbated by factors such as the COVID-19 pandemic, undermines the effectiveness of care management programs and negatively impacts both subject well-being and healthcare costs.

### SUMMARY

Some aspects of the present disclosure relate to techniques for generating a structured output based on one or more unstructured portions of electronic health record (EHR) data of a subject. The one or more unstructured portions may correspond to document refences or binary files comprising raw data including, but are not limited to, visit notes, prescription requests, lab results, immunizations, procedures, medications, faxes, visit note records, demographic information, medical conditions, prior medical observations, questionnaires, referrals, discharge summaries, imaging results, consent forms, nursing notes, patient education materials, administrative forms, scheduled encounters or the like. The disclosed techniques may include accessing the one or more unstructured portions of the EHR data of the subject via an application programmable interface (API), (for example) a long record API.

The long record API may retrieve the one or more unstructured portions from an EHR or a database based on a request from a user (for example) a care manager, a care manager supervisor, a clinician, a medical provider, or an entity facilitating medical monitoring or treatment for the subject. The request may comprise identifiers corresponding to the subject that may include, but are not limited to, a subject identifier (ID), a population ID or encounter date (e.g., a reference date corresponding to the recent communication event or last communication date). The request may include generating a result using the one or more GenAI models based on the one or more unstructured portions.

The one or more unstructured portions may be filtered based on a set of filtering criteria defined within the long record API. The set of filtering criteria may apply specific logic to exclude unstructured portions with keywords such as “form”, “education”, “edu”, “questionnaire”, “referral”, “prescription letter”, or any other “letter” e.g., from the associated titles. In some aspects, the one or more unstructured portions related to specific tests, orders, response forms or other such categories may be excluded, resulting in filtered data. The filtered data may retain only relevant and actionable data for further processing.

In some aspects, the retrieval of the one or more unstructured portions may be governed by the set of filtering criteria, which may serve to reduce long record API usage. The long record API may retrieve the subject profile based on the provided identifiers (for example) the subject ID and the population ID. Additionally, if the last communication is specified, the one or more unstructured portions associated with encounters occurred up until a recent communication event may be retrieved. In some instances, if the last communication may not be specified, the long record API (by default) may retrieve the one or more unstructured portions associated with the encounters from a specific look-back period. In some aspects, the look-back period may be 3 months. In some aspects, the look-back period may be other than 3 months. In some aspects, the long record API may fetch additional information (e.g., relevant data elements within the one or more unstructured portions) associated with the encounters from the EHR or the database based on a request (e.g., to generate a summary). One or more API calls may be made to fetch the additional information including (for example) observations, medications, procedures, immunizations etc. in filtered form.

The filtered data, comprising (for example) the relevant data elements or the one or more unstructured portions, may be transformed into a long record data based on a field-value clustering. Field-value clustering may be employed to associate discrete, unstructured data entities (e.g., medications, conditions, procedures, observations or the like) within the one or more unstructured portions with specific events including encounters or procedures, even when such associations may not be explicitly defined in the raw binary files or the one or more unstructured portions. Such a process may create meaningful relationships between the data entities, which may then be presented in a semi-structured long record data view. In some aspects, the long record data view comprising the long record data may be a JSON object. In some other aspects, the long record data may be presented in other formats (for example) xlsx, xml, csv, or the like.

In some aspects, the long record data may be processed to decode binary data (e.g., base64) into a text-readable format (e.g., a Unicode transformation format-8 bit or UTF-8). The base64 encoded data may be converted into its original binary form and then into UTF-8. Additionally, the text-readable format may be stripped to remove tags, scripts, and other non-text elements. Other non-relevant data elements including embedded multimedia content (e.g., images, audio, or video tags), inline cascading style sheets (CSS), or comment sections may also be stripped to convert the text-readable format or data into text-based format (e.g., plain text). In some aspects, if the long record data may exist in a text-readable format (for example) a JSON object, the long record data may be processed (e.g., stripped) into a text-based format (e.g., a plain text).

The text-based format may be transformed into a structured output using one or more GenAI models. The one or more GenAI models may generate a prompt based on the plain text, extract data elements from the plain text based on the prompt and process the data elements to generate a structured output. In some aspects, the prompt may be generated using an ontology that may provide structured knowledge of domain-specific terms and their interrelationships. The ontology may help providing prompt alignment with the relevant context including (for example) medical concepts. Based on the generated prompt, the plain text may be processed to automatically extract one or more data elements. In some aspects, a large language model (LLM), such as GPT or Cohere Command R. may be used to extract the one or more data elements. In some aspects, the one or more data elements may include, but are not limited to, visit rationales or follow-up instructions.

The structured output (e.g., summary) may be generated using a machine-learning technique e.g., few-shot learning, where the prompt may instruct the LLM to extract the one or more data elements based on (fewer) synthetic examples. The synthetic examples may be customized for different use cases, user types or the like. In some aspects, a single prompt may be used to extract the data elements. In some other aspects, extractions may be performed multiple times (e.g., in parallel or at different time points), using different prompts that may request the extraction of distinct types of information in the form of data elements. The one or more data elements may be processed to generate the structured output into a template (e.g., a predefined format) using a set of transformation criteria. The set of transforming criteria may include (for example) logic requirements and format requirements.

The structured output may comprise a summary, providing a high-level overview of the health status of the subject. In some aspects, the summary may capture incremental details of a subject from the last successful communication to the time when the summary may be generated. The summary may include one or more of: subject profile (e.g., name, age, conditions, allergies), care team, clinical summary associated with encounters (e.g., medical history, diagnoses, treatments, lab results, medications), social factors (e.g., access to transportation, money and resources, family and home situation), future appointments (e.g., scheduled follow-ups, referrals to specialists etc.) or a combination thereof. Additionally, the structured output may include supporting facts that provide detailed data to substantiate the summary, often presented in a tabular format, including, but are not limited to, lab results, medical history, diagnoses, medications, treatments, and timelines of key events (e.g., surgeries, hospitalizations). The supporting facts may also encompass granular information including the dates associated with specific medical events, diagnoses, or procedures.

The structured output may be displayed on the interface associated with the user as a result of the user request. In some aspects, the result may be other than the structured output (e.g., the summary). Other results may include (for example) talking points (e.g., scripted prompts for care managers who prefer phone calls), call activity logs (e.g., calls made, call summaries, interactions with the patient), care plan updates (e.g., modifications to treatment plans, setting of health goals), reminders (e.g., scheduled tasks, follow-ups for users), longitudinal plan (e.g., goals and interventions, personalized for each subject), longitudinal record (e.g., customized summaries for defined look-back periods, tailored to specific care programs), outreach messages (e.g., personalized communication for enrolling subjects in care management programs), care handoff documentation (e.g., transition documents for pediatric subjects aging into adult care) etc. It will be appreciated that techniques disclosed herein may reduce hallucinations (e.g., via intelligent prompt engineering and data processing), thereby improving the accuracy of the result generated.

The result displayed on the interface may be scored by the user using interactive elements including (for example) approval or disapproval icons, editable text fields, or a numerical scoring system to gauge user satisfaction with the result. In some aspects, the user score may serve as a feedback to refine the result or be used for training the one or more GenAI models. Based on a user score (e.g., if a result receives a low score) the underlying prompt or summary examples may be adjusted. In some aspects, the one or more data elements may be assigned user scores. In some other aspects, the result may be assigned user scores before part or all of the result may be sent over to the subject for a communication purposes. The user scores may represent a review or validation of the accuracy of the one or more data elements, structured output, or any other result by the user. For instance, a result (e.g., an outreach message) may be presented to the user for scoring before being forwarded to the subject.

The validation and reviewing process of the result by the user may be automated using an auto-evaluator. The auto-evaluator (e.g., another LLM) may be used to assign evaluation scores to the one or more data elements, the result generated by the one or more GenAI models. The auto-evaluator may compare the result or the one or more data elements with a baseline dataset (for example) an annotated data to assign the evaluation scores. User annotators may annotate the text-based format or the plain text using an annotation tool. In some aspects, an automated annotation and review tool (ART) may be used to annotate the plain text. The annotation tool may generate annotations comprising (for example) an assigned label that may be designated to a data element present within the plain text and a location of the designated data element associated with the assigned label. For instance, a “condition” label may be assigned to ‘diabetes,’ and a “medication” label may be assigned to ‘aspirin,’ with each label pointing to its respective location within the plain text. The annotations may be compared with the plain text to identify the data elements that may align with the annotations and enrich the plain text with the annotations to generate the annotated data. In some aspects, a separate JSON object may be generated based on the enrichment, which may serve as the annotated data to be used as the baseline dataset to assign evaluation scores.

The auto-evaluator may reach a confidence threshold before the evaluation scores may be used for rapid iteration and validation. To reach a confidence threshold, the user scores and the evaluation scores may be compared, generating a threshold. In some aspects, the threshold may be calculated as a performance metric (for example) an error margin or confidence level. The threshold may be fed back into the auto-evaluator to adjust the parameters of the auto-evaluator and reduce discrepancies between evaluation scores and user scores. The iterative process continues until a confidence level (e.g., 90% or above) may be achieved, indicating reliable evaluation. Once the confidence threshold is reached, the auto-evaluator may automatically assess the results, saving time by eliminating the need for manual scoring and intervention. Scoring using an LLM (e.g., an auto-evaluator) may enhance efficiency, consistency, scalability, and continuous improvement, while enhancing resource allocation, and ensuring quicker and more accurate evaluations.

In some aspects, the evaluation scores may be represented as a single aggregated score assigned to the one or more data elements or the result. In some other aspects, the evaluation scores may be assigned on a per-element basis, where each individual data element of the one or more data elements may receive its own evaluation score. The evaluation scores may reflect how well each part of the output of the one or more GenAI models (e.g., the one or more data elements or the result) aligns with the annotated data. In some aspects, the generated evaluation scores may be used in conjunction with a loss function or objective function to facilitate the training of the one or more GenAI models.

The result generated using the one or more GenAI models may reduce manual tasks including (for example) chart reviews and documentation by up to 75%, automating updates to care plans and medical histories of subjects. Such efficiency may enable users to manage more subjects, improving productivity and subject support. The one or more GenAI models may also decrease emergency department utilization and inpatient admissions, increase care management program enrollment, and reduce costs while improving subject outcomes. Ultimately, such automated workflow may empower users to focus more on meaningful subjects and less on administrative tasks.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates an exemplary overview of implementing a strategic content reduction to facilitate user communications based on electronic health record (EHR) data of a subject in accordance with some aspects of the present disclosure.

FIG. 2 shows an exemplary illustration of processing one or more unstructured portions derived from the EHR data of the subject in accordance with some aspects of the present disclosure.

FIG. 3 illustrates an exemplary unified modeling language (UML) diagram of field-value clustering based on association with a specific encounter to generate long record data in accordance with some aspects of the present disclosure.

FIG. 4 illustrates an exemplary implementation of transforming a plain text into a result using one or more generative artificial intelligence (GenAI) models in accordance with some aspects of the present disclosure.

FIG. 5A illustrates an exemplary process for evaluating and training the one or more GenAI models based on evaluation scores generated by an auto-evaluator in accordance with some aspects of the present disclosure.

FIG. 5B illustrates an exemplary process for training the auto-evaluator based on a threshold in accordance with some aspects of the present disclosure.

FIG. 6 illustrates an exemplary sequence diagram for extracting and structuring one or more unstructured portions into the structured output using business logic and the one or more GenAI models in accordance with some aspects of the present disclosure.

FIG. 7 illustrates an exemplary healthcare portal of a subject within a healthcare management platform in accordance with some aspects of the present disclosure.

FIG. 8A illustrates an exemplary user interface that the user may experience when accessing a summary prior to its generation within the healthcare management platform, in accordance with some aspects of the present disclosure.

FIG. 8B illustrates an exemplary user interface featuring a summary generation within the healthcare management platform in accordance with some aspects of the present disclosure.

FIG. 8C illustrates an exemplary segment of at least a portion of the structured output accessed by a link in accordance with some aspects of the present disclosure.

FIG. 9 illustrates an example flowchart of implementing the strategic content reduction to facilitate the user communications in accordance with some aspects of the present disclosure.

FIG. 10 depicts a simplified diagram of a distributed system for implementing an aspect in accordance with some aspects of the present disclosure.

FIG. 11 is a simplified block diagram of a cloud-based system environment in accordance with some aspects of the present disclosure.

FIG. 12 illustrates an exemplary computer system that may be used to implement certain aspects of the present disclosure.

### DETAILED DESCRIPTION

The present disclosure relates to techniques that leverage one or more generative artificial intelligence (GenAI) models to transform electronic health record (EHR) data of a subject, often including (for example) unstructured portions, into succinct and structured representations configured to enhance effective communication between users and subjects. Such transformations may provide structured representations that support quicker and more effective communication between user endpoints (e.g., a computing system or device associated with a user such as care manager, a care manager supervisor or a medical specialist) and a third-party device or a subject endpoint (e.g., a computing system or device associated with the subject). For instance, the transformation may provide a filtration and/or representation of information that may support automation of part, or all of a workflow (e.g., reducing or eliminating data collection, data filtering, data synthesis, data structuring, etc.) thereby reducing burden of manual tasks.

In some aspects, the disclosed techniques may produce transformed content (e.g., a structured output or other result) that supports communications more likely to lead to successful engagements and/or outcomes with a third party (e.g., subject). Such outcomes may include fewer or no emergency department visits, reduced hospitalizations, healthier births, and more successful recoveries following injuries or surgeries. The structured output may efficiently represent longitudinal data including, but not limited to, encounters, procedures, conditions, observations, static data (e.g., immunizations), supporting facts (e.g., vitals, lab results, medical history, diagnoses, medications, treatments, and timelines of key events) etc. The techniques, as disclosed herein, may improve the time efficiency of users (e.g., to three-folds or more or in some examples even reducing a 45-minute review—that may include assessing a dozen or more separate files—down to a 5-minute review by accessing only the structured output).

According to some aspects of the present disclosure, the structured output may comprise a summary that provides a high-level overview of the health status of a subject, capturing incremental details of the subject from the last successful communication to the time when the summary may be generated. The disclosed techniques may generate a summary based on a template (e.g., predefined format) that may include, but is not limited to, subject profile (e.g., name, age, conditions, allergies), care team, clinical summary associated with encounters (e.g., medical history, diagnoses, treatments, lab results, medications), social factors (e.g., access to transportation, money and resources, family and home situation), future appointments (e.g., scheduled follow-ups, referrals to specialists etc.) or a combination thereof. The summary may be substantiated by supporting facts that provide additional details to validate or elaborate on the information in the summary. Some aspects of the present disclosure may achieve successful outcomes, whether in absolute terms or compared to existing outcomes, even when a subject's circumstances or medical history may be complex or abnormal.

Further, the disclosed techniques may produce other structured representations or results including, but are not limited to, talking points (e.g., scripted prompts for care managers who prefer phone calls), call activity logs (e.g., calls made, call summaries, interactions with the patient), care plan updates (e.g., modifications to treatment plans, setting of health goals), reminders (e.g., scheduled tasks, follow-ups for users), longitudinal plans (e.g., goals and interventions, personalized for each subject), longitudinal records (e.g., customized summaries for defined look-back periods, tailored to specific care programs), outreach messages (e.g., personalized communication for enrolling subjects in care management programs), care handoff documentation (e.g., transition documents for pediatric subjects aging into adult care) etc.

The structured representations may result in reduced time expenditure, regarding user time and/or resource usage involved in outreach communication or appointment scheduling. Further, the disclosed techniques may decrease emergency department utilization and inpatient admissions for subjects under a care management program, increase enrollment in care management programs, and improve subject outcomes, thereby reducing costs for the subjects. Such efficiency gain may enable users to manage more subjects in less time, improving productivity and providing better subject support. It will be appreciated that techniques disclosed herein may reduce hallucinations to improve the accuracy of the generated structured representations or results.

A result may be generated based on EHR data of a subject comprising one or more unstructured portions that may be accessed via a long record application programmable interface (API). The long record API may retrieve the one or more unstructured portions associated with the EHR data of a subject from an EHR or a database based on a request from a user (for example) a care manager, a care manager supervisor, a clinician, a medical provider, or an entity facilitating medical monitoring or treatment for a subject. The user may request via an interface (herein after referred to as user interface (UI)) associated with the user endpoint to generate a result based on the one or more unstructured portions. The request may comprise identifiers corresponding to the subject that may include, but are not limited to, subject ID, population ID or encounter date (e.g., a reference date corresponding to the recent successful communication event or last communication date). In some aspects, the subject may have had one or more encounters after the last communication date.

According to some aspects of the present disclosure, the user may retrieve one or more unstructured portions from the EHR, based on the request. The one or more unstructured portions may correspond to document refences or binary files comprising raw data including, but are not limited to, visit notes, prescription requests, lab results, immunizations, procedures, medications, faxes, visit note records, demographic information, medical conditions, prior medical observations, questionnaires, referrals, discharge summaries, imaging results, consent forms, nursing notes, patient education materials, administrative forms, scheduled encounters or the like. The long record API may filter the one or more unstructured portions based on a set of filtering criteria (herein after referred to as filtering criteria) defined within the long record API. Based on the filtering criteria, the one or more unstructured portions related to specific tests, orders, response forms or other such categories may be excluded. The set of filtering criteria may apply a logic to exclude portions including keywords (for example) “form”, “education”, “edu”, “questionnaire”, “referral”, “prescription letter”, or any other “letter” from the associated titles. Using such criteria, filtered data may be generated accordingly. It will be appreciated that some of the disclosed techniques may focus on the pertinent clinical information while excluding extraneous or non-clinical content.

According to some other aspects of the present disclosure, the retrieval of the one or more unstructured portions may be governed by the set of filtering criteria, which may serve to reduce long record API usage. The long record API may retrieve the subject profile based on the provided identifiers (for example) subject ID and population ID. The subject profile include (for example) name, age, sex, conditions (e.g., diagnosis and symptoms) or allergies. Additionally, if the encounter date is specified, the one or more unstructured portions associated with encounters up until the recent communication event may be retrieved, else the long record API may retrieve the unstructured portions from encounters within a default look-back period. In some aspects, the look-back period may be 3 months. In some aspects, the look-back period may be other than 3 months. Some aspects of the present disclosure relate to techniques for fetching additional information (e.g., relevant data elements within the one or more unstructured portions) associated with the encounters from the EHR or the database based on the user request (e.g., to generate a structured output). One or more API calls may be made by the long record API to fetch the additional information including (for example) observations, medications, procedures, immunizations etc. in filtered form.

Some of the disclosed techniques may transform the filtered data into a long record data based on a clustering technique (for example) a field-value clustering. Field-value clustering may be employed to associate discrete, unstructured data fields (e.g., medications, conditions, procedures, observations or the like) within the one or more unstructured portions with specific encounters, even when such associations may not be explicitly defined in the raw binary files or the one or more unstructured portions. In some aspects, field-value clustering may create meaningful relationships between the data fields, which may then be presented in a semi-structured long record data view. The long record data may include, but is not limited to, encounter details (e.g., reason for visit, arrival time, discharge time, care period start/end, participants, status, type, and priority) and procedure information (e.g., procedure code, start time, and related providers). The long record data view may be a tabular representation of the semi-structured long record supporting facts, which may not conform to a strict schema, but still comprise of some level of organization including tags or key-value pairs. In some aspects, the long record data view, comprising the long record data, may be a JSON object. In some other aspects, the long record data may be presented in other formats (for example) xlsx, xml, csv, or the like.

Some techniques of the present disclosure relate to processing the long record data into a plain text by decoding the encoded data and/or stripping of unnecessary elements using a decoding module. In some aspects, the decoding module may decode the long record data in binary format (e.g., base64) into a text-readable format (e.g., a Unicode transformation format-8 bit or UTF-8). Additionally, the text-readable format may be stripped to remove tags, scripts, and other non-text elements. For instance, embedded HTML tags (for example)<LR concepts>, <supporting facts> and scripting elements including <script>, <style>, and JavaScript functions may be removed. Other non-relevant data elements including embedded multimedia content (e.g., images, audio, or video tags), inline cascading style sheets (CSS), or comment sections may also be stripped to convert the text-readable format or data into text-based format (e.g., plain text). In some aspects, if the long record data may exist in a text-readable format (for example) a JSON object, the long record data may be processed (e.g., stripped) into a plain text.

The plain text may be transformed into a structured output using a GenAI service that may access one or more GenAI models. The one or more GenAI models may comprise of a prompt generator, a large language model (LLM) to extract data elements and a processing module. The prompt generator may be employed to generate a prompt based on the plain text, by applying prompting techniques including (for example) chain of thought (Cot), personification, structured output, reflection, separation of concerns or a combination thereof. Such techniques may ensure that the prompt effectively captures the context of the plain text. In some aspects, the prompt may be generated using an ontology that may provide structured knowledge of domain-specific terms and their interrelationships. The ontology may ensure that the prompt aligns with the relevant context including (for example) medical concepts.

Based on the generated prompt, the plain text may be processed by the LLM to automatically extract one or more data elements from the plain text. In some aspects, the LLM may utilize model (for example) Cohere Command R to automatically extract the data elements. In some aspects, the one or more data elements may include, but are not limited to, visit rationales and/or follow-up instructions. In some other aspects, the one or more data elements may include additional information other than the visit rationales and/or the follow-up instructions. In some aspects, the data elements may be extracted synchronously to reduce latency of the LLM. In some aspects, a single prompt may be utilized to extract the relevant data elements. In some other aspects, extractions may occur iteratively, either concurrently or at different time points, with different prompts designed to request the extraction of distinct types of information as data elements. In some instances, prompts may be generated to further or alternatively cause the LLM to draft talking points for a user to consider and/or a summary of a recent conversation between the user and a subject.

The one or more data elements may be processed by the processing module to generate a result in a natural language format. In some aspects, the result may be provided in rich text format (rtf), which may enable inclusion of various formatting options including bold, italics, underlining, and the ability to insert images and other elements. In some aspects, the processing module may strategically reduce the content of the structured output. According to some of the disclosed techniques, the result may be processed based on a set of transformation criteria including (for example) logic requirements and format requirements. The logic and format requirements may ensure consistent representation by applying specific rules for inclusion, exclusion, and formatting including, but are not limited to, capitalizing names, listing medications with dosages, excluding non-normal lab results, using a bullet format for conditions and allergies or the like.

The result generated by the one or more GenAI models may be provided to the user via the UI to assist in reviewing, validating, or further refining the information. The user may assign scores (herein after referred to as user scores) that may serve as feedback to refine the result or be used for training the one or more GenAI models. In addition to the result, the one or more data elements may be assigned user scores, according to some aspects of the present disclosure. In some aspects, the result may be assigned user scores before part or all of the result may be sent over to the subject. The user scores may be assigned using interactive elements including (for example) approval or disapproval icons, editable text fields, or a numerical scoring system to gauge user satisfaction with the result. The user scores may represent a review or validation of the accuracy of the result by the user.

Some of the disclosed techniques may relate to automating the review or validation process of the result or the one or more data elements by using an auto-evaluator. The auto-evaluator may generate an evaluation score for the result or the one or more data elements by comparing the result or the one or more data elements with a baseline dataset or a test dataset. The baseline dataset may help the auto-evaluator to understand the expected output and evaluate the extraction capabilities of the LLM or the one or more GenAI models. The baseline dataset may comprise of annotated data, generated, or augmented with user input, to assess the accuracy and reliability of the extracted data elements or results.

In some aspects, the user may annotate the plain text using an annotation tool. The annotation tool may generate annotations comprising (for example) an assigned label that may be designated to a data element (e.g., name of the subject, age, medical condition, visit date, symptoms, prescribed medications etc.) present within the plain text and a location of the designated data element associated with the assigned label. An annotation processor may compare the annotations with the plain text to identify the data elements that may align with the annotations and enrich the plain text with the annotations to generate the annotated data. In some aspects, a separate JSON object may be generated by the annotation processor based on the enrichment, which may serve as the annotated data to be used as the baseline dataset to assign evaluation scores. Evaluation may be essential to ensure that the output of the one or more GenAI models may meet expectations of the user and deliver precise, reliable results.

Further, some of the disclosed techniques may relate to the application of the auto-evaluator once the auto-evaluator may reach a confidence threshold. To reach the confidence threshold, the auto-evaluator may be iteratively trained using a threshold, which may be generated based on a comparison of the user scores and the evaluation scores. In some aspects, the threshold may be calculated as a performance metric (for example) an error margin or confidence level. Once the confidence threshold may be reached, the auto-evaluator may automatically assess results, saving time by eliminating the need for manual scoring and intervention. A confidence threshold may refer to a specific level of certainty, often expressed as a percentage (e.g., 90% or above).

In some aspects, the evaluation scores may be represented as a single aggregated score assigned to one or more data elements or a result. In some other aspects, the evaluation scores may be assigned on a per-element basis, where each individual data element of the one or more data elements may receive its own evaluation score. Automated scoring may improve efficiency, consistency, and scalability, driving continuous improvements that may enhance resource utilization and ensure faster, more precise evaluations. The generated evaluation scores may be used in conjunction with a loss function or objective function to facilitate the training of the one or more GenAI models. Such automated workflow may enable users to focus on high-value tasks, driving better outcomes while minimizing time spent on administrative overhead.

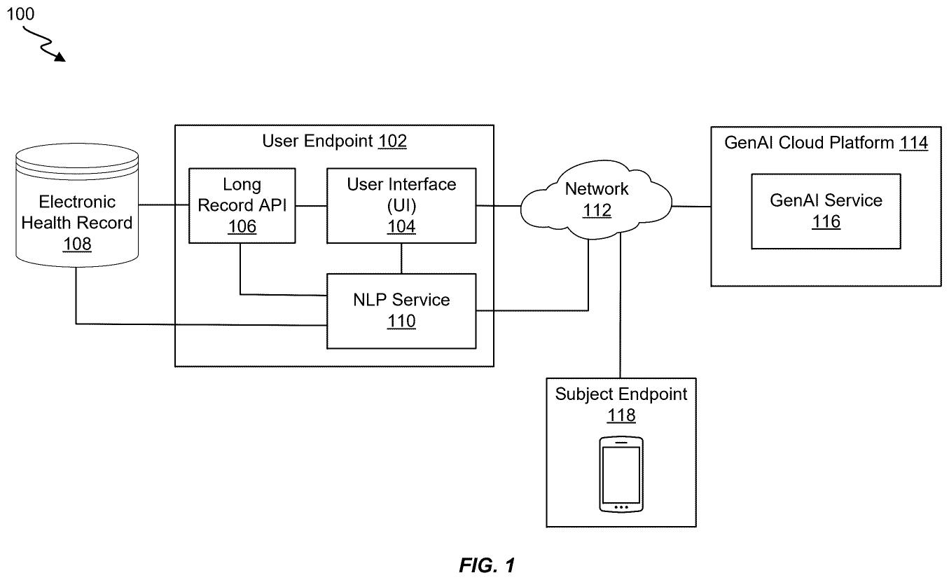

FIG. 1 illustrates an exemplary overview 100 of implementing a strategic content reduction to facilitate user communications based on electronic health record (EHR) data of a subject in accordance with some aspects of the present disclosure. Exemplary overview 100 comprises a user endpoint 102, a subject endpoint 118, a network 112, a GenAI cloud platform 114, and an EHR 108 or a database. The user endpoint 102 and the subject endpoint 118 may include a mobile device, a tablet, a laptop, a desktop computer, a computer server etc. The user endpoint 102 may run an application, a web-based application or a cloud-app and may provide a user interface (UI) 104.

A care manager, a care manager supervisor, a clinician, a medical provider, or an entity facilitating medical monitoring or treatment for a subject (which may collectively or individually be referred to as a user) may interact with the UI 104 of the application to generate, review, store and assess records of a subject. The user may utilize the UI 104 to send messages to the subject endpoint 118, and read and respond to messages from the subject endpoint 118 registered on the application, thereby facilitating communication within the organization. The UI 104 may represent an application interface authorized and registered for use within a specific territory, potentially with restricted access for other registered individuals. The UI 104 may be a web-based application. In some aspects, the UI 104 may be a dedicated application featuring a custom-designed graphical user interface (GUI), for example, EpicCare™, PowerChart™ or MessageCenter™.

The user may store recently created EHR data (e.g., documents or medical notes) or retrieve the EHR data from the database or EHR 108. The user endpoint 102 may include components to receive information related to selected records of the subject or other subjects based on a request via the UI 104. For instance, the UI 104 may put a request and a long record application programmable interface (API) 106 may retrieve EHR data associated with a subject based on the request. The request may comprise of identifiers corresponding to a subject that may include subject ID, population ID or encounter date. The encounter date may be a reference date corresponding to the recent successful communication event with the subject. The request may further specify to generate a result based on the one or more unstructured portions. In some aspects, one or more unstructured portions (e.g., medical notes and/or part or all of an EHR data) may be retrieved utilizing the long record API 106, based on the request. In some aspects, the retrieval may be based on filtering criteria defined within the long record API that may reduce data volume and minimize API usage.

The NLP service 110 and the UI 104 may be communicatively coupled to the long record API 106. In some aspects, the NLP service 110 may access selection criteria to select specific unstructured portions of the EHR data of the subject retrieved by the long record API 106. The NLP service 110 may be employed to identify the content and intent of the one or more unstructured portions and decode the one or more unstructured portion into a text-based format (e.g., a plain text). The NLP service 110 may be comprised of a software application or set of applications, programs, routines, or computer performed services. The NLP service 110, along with the long record API 106, may reside on the computing device of the user such as laptop, smartphone, and the like. In some aspects, the NLP service 110 and the long record API 106 may reside on a remote server communicatively coupled to the computing device of the user. In some other aspects, the NLP service 110 may comprise an application or set of applications deployed in a cloud-based platform providing virtualized resources, for example, OCI, AWS, and Google cloud.

The EHR 108 may be capable of storing multiple records (e.g., the EHR data) for one or more subjects and may consist of various data storage devices distributed across multiple computers and/or servers. The EHR 108 system may encompass one or more specialized EHR systems, such as the EHR systems used in hospitals, health information exchanges, clinical genetics/genomics, ambulatory clinics, psychiatry, or neurology practices, as well as systems for managing insurance, collections, or claims records. In some aspects, the EHR 108 may incorporate one or more data repositories for health-related records, along with computers or servers that manage the storage and retrieval of such records. In some aspects, the EHR 108 or database may also be implemented as a cloud-based platform or distributed across several physical locations. In some aspects, the EHR 108 may comprise of systems to capture and store dynamic health data from sources including mobile health applications, telemedicine platforms, or remote patient monitoring tools. In some other aspects, the EHR 108 may comprise of multiple data storage units distributed across a network of interconnected computers and servers for better scalability, fault tolerance and efficient data retrieval.

The user endpoint 102 may utilize the network 112 to communicate with the GenAI cloud platform 114 and the subject endpoint 118. The network 112 may encompass a wide range of communication infrastructures including both public and private networks, as well as various devices including switches, routers and firewalls, all enabling efficient information exchange and connectivity across multiple nodes. In some aspects, the network 112 may be a collection of interconnected devices (for example) computers, servers, and routers, communicating with each other, enabling data exchange and resource sharing. The network 112 may be a local area network (LAN), designed for high-speed communication within a limited geographic area, typically utilizing Ethernet or Wi-Fi connections. Alternatively, the network 112 may extend to a wide area network (WAN) that links multiple LANs over larger distances, or a metropolitan area network (MAN) covering networks within specific regions such as hospitals, corporate setups etc. Other form of the network 112 may include campus area networks (CAN), storage area networks (SAN), and virtual private networks (VPN), which may provide secure and encrypted communications over broader public networks (e.g., the internet). However, the selection of network 112 does not limit the scope of the disclosure, as it may vary based on factors such as scalability, security, and performance requirements.

The GenAI cloud platform 114 comprises a GenAI service 116 that may utilize the one or more GenAI models to transform the plain text present in the text-based format into a result. According to some aspects of the present disclosure, executing the GenAI service 116 may refer to executing the one or more GenAI models through cloud service providers, offering pre-built models, APIs, and infrastructures. In other instances, the GenAI service 116 may include customized GenAI models or solutions that may be trained on propriety data or domain-specific data. In some aspects, the one or more GenAI models may incorporate large language models (LLMs) to enhance processing and content generation. A framework (e.g., LangChain) may be utilized to build the one or more GenAI models, enabling efficient prompt engineering and seamless integration with various data sources and interfaces including the user endpoint 102 associated with the users or other relevant systems.

The GenAI service 116 may provide access to the one or more GenAI models that may generate content based on the patterns or information learned from the training data or data provided at run-time. In some aspects, the one or more GenAI models may access the plain text forwarded by the NLP service 110 over the network 112 and extract one or more data elements from the provided plain text based on intelligent prompt engineering and data processing. The extracted data elements may then be processed and strategically reduced, adhering to a set of transformation criteria designed to define the format of the result (e.g., a structured output).

The structured output may represent (for example) strategically reduced content that captures incremental details of a subject including, but are not limited to, trends in medical encounters over time, medication history, medication intake versus symptoms severity, abnormal vitals, triggered allergies, and/or other relevant data points. The structured output comprises a summary that may be produced based on a template using the one or more GenAI models. In some aspects, the GenAI service 116 may be used to draft talking points for the user to consider, or to provide a summary of a recent conversation between a user and a subject. In some aspects, the GenAI service 116 may process communications received from the subject via the subject endpoint 118, evaluating or processing the communications before incorporating it into the structured output. The communications may also be used for monitoring subject progress, identifying patterns, generating alerts for time-sensitive actions, and assisting in updating care plans. In some other aspects, the GenAI service 116 may be utilized to generate reminders or suggest alternative options if a subject fails to show up for a scheduled medical encounter, ensuring continuity of care and enhancing the decision-making process for the user. The result may be sent to the user via the UI 104 to assist the user. After review or validation by the user, part or all of the result may be forwarded to the subject endpoint 118 over the network 112 for communication and follow-up actions.

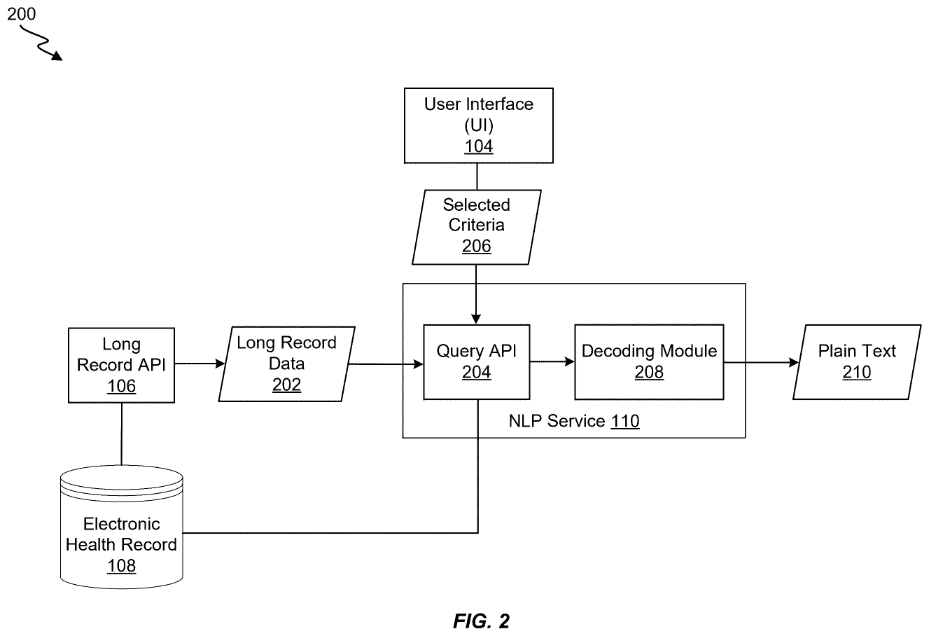

FIG. 2 shows an exemplary illustration of processing one or more unstructured portions derived from the EHR data of the subject in accordance with some aspects of the present disclosure. The long record API 106 may process the one or more unstructured portions of the EHR data to efficiently filter and extract long record data 202 based on the filtering criteria. The long record API 106 may serve as a software interface that enables applications to retrieve and interact with the EHR data of a subject across multiple sources. In some aspects, the long record API 106 may facilitate the separation of structured portions (e.g., tabular data or coded information) from unstructured portions (e.g., free-text notes, medical record notes, images, or scanned documents). The long record API 106 may utilize parsing algorithms, NLP tools, machine learning techniques or a combination thereof, to separate the portions.

According to some aspects of the present disclosure, the long record API 106 may retrieve one or more unstructured portions from the EHR 108 based on the request of the user. The one or more unstructured portions may include binary files having a binary format that may preserve the entirety of the one or more unstructured portions including textual content, images, or any embedded multimedia elements, in their original state. In some aspects, the binary files may be retrieved synchronously to reduce retrieval latency of the long record API. The binary files may include data pertaining to (for example) a subject's demographic data, notes, faxes, medical condition, prior medical observations, document references, medical encounters, medications, immunizations, procedures, questionnaires, referrals, discharge summaries, imaging results, consent forms, nursing notes, patient education materials, administrative forms, scheduled encounters, etc. The binary files may also represent or be indicative of relationships between different data types or fields (e.g., which medications were prescribed to a subject when the subject underwent a given procedure).

Content of the binary files or the one or more unstructured portions may be filtered according to the filtering criteria defined within the long record API 106. The long record API 106 may selectively process relevant binary files based on such predefined filtering criteria, which may include conditions to exclude certain types of records and note categories from the binary files or the one or more unstructured portions. Such exclusions may be based on the type of data, title, and the nature of the content. Specifically, any portion categorized as a “form”—including, but not limited to, patient care forms, assessment forms, imaging exam information forms, and various administrative and procedural forms—may be excluded. Subject education materials including inpatient and outpatient education, may also be filtered out. Questionnaires, referrals (e.g., endoscopy referrals), prescriptions (e.g., prescription requests), letters (such as patient letters or patient results letters), and nursing notes (including nursing notes from emergency departments or general nursing documentation) may be excluded from consideration. The filtering criteria apply a logic to filter out records including keywords such as “form”, “education”, “edu”, “questionnaire”, “referral”, “prescription letter”, or any other “letter” from the associated titles. Furthermore, additional filtering may be implemented to exclude unstructured portions related to specific tests, orders, response forms, and other predefined categories, which may ensure that only the relevant and actionable data may be retained for further processing. The filtering criteria may enable a streamlined process, focusing on pertinent clinical information while excluding extraneous or non-clinical content.

According to some other aspects of the present disclosure, the retrieval of the one or more unstructured portions may be governed by the filtering criteria, which may serve to reduce long record API 106 usage. The long record API 106 may retrieve subject profile information (e.g., name, age, sex, conditions, or allergies) and encounter details (e.g., within a default look-back period or up until the recent communication event), based on provided identifiers such as subject ID, population ID, or encounter date. In some aspects, the look-back period may be 3 months. In some other aspects, the look-back period may be other than 3 months. In some instances, additional information (e.g., relevant data elements within the one or more unstructured portions) associated with the encounters may be retrieved from the EHR or the database based on the user request (e.g., to generate a summary). The additional information comprises binary files including (for example) conditions, procedures, immunizations, medications etc. The subject profile along with encounter details and/or the additional information may be aggregated to form the long record data 202.

In some aspects, the long record API 106 may associate values of discrete fields within a binary file that may otherwise be unassociated to generate the long record data 202. For instance, the long record API 106 may use field overlap, correlation assessment and/or other techniques to make associations between data fields within the one or more unstructured portions including linking medications with corresponding procedures, aligning diagnoses with encounters, or correlating a subject's medical history with the subject's current treatment plans. In some aspects, the long record data 202 may be stored in the EHR 108.

Some aspects of the present disclosure relate to querying the long record data 202 by a query API 204 based on selected criteria 206. The user may select or choose criteria from the various selection criteria available on the UI 104. In some aspects, the selected criteria 206 may include time-based filters (e.g., focusing on a specific date range or a lookback period (e.g., 3 months)) to review the long record data 202 related to a particular timeframe. In some other aspects, the selected criteria 206 may filter by the type of medical encounter including outpatient visits, emergency department visits, in-patient procedures etc., to narrow down the long record data 202. Additionally, the selected criteria 206 may further be based on, but are not limited to, specific diagnoses, conditions, symptom severity, clinical observations, medication-related information, care plans, or other relevant factors. In some aspects, additional information or binary files related to the long record data 202 may be retrieved from the EHR 108 based on the selected criteria 206. The query API 204 may enable the extraction of further relevant information associated with the long record data 202, facilitating a more comprehensive analysis and a better understanding of the data for clinical decision-making. Output from the query API 204 may be forwarded to a decoding module 208 present within the NLP service 110.

According to some other aspects of the present disclosure, the long record data 202 may be forwarded to the decoding module 208. The decoding module 208 may be responsible for transforming data that may have been encoded into a text-readable format, typically Base64, into its original binary form. Once decoded, the binary data, typically represented as a string of characters, may then be converted into a Unicode transformation format 8-bit (UTF-8) encoding. For instance, the decoding module 208 may leverage technologies (for example) batch processing, parallel computing, or specialized libraries (e.g., OpenSSL or Base64 decoding algorithms) to efficiently decode base64 encoded data into UTF-8 encoded text. Such conversion may ensure that the textual content may be accurately represented, preserving special characters and symbols, and enables further processing, analysis, or display in a standardized and text-readable format. By using UTF-8 encoding, the NLP service 110 may ensure compatibility across various systems and maintain the integrity of the original information in the text-readable form.

Further, the decoding module 208 may strip any embedded HTML tags (e.g., <div>, <Heuristics>, <LR Concept> etc.), scripts (e.g., JavaScript, cascading style sheets (CSS), embedded multimedia elements etc.), or other non-text elements that may have been included in the long record data 202. Stripping HTML may help eliminate formatting, embedded images, and links, resulting in a clean and simplified text-based format (e.g., plain text) of the long record data 202. In some aspects, the long record data 202 may directly be stripped by a decoding module 208. It will be appreciated that, in some instances, the long record data 202 or the queried long record data includes multiple segments (e.g., pertaining to multiple binary files, data types, associated dates, etc.). Each segment corresponding to a given subject may be separately decoded and/or stripped of HTML, either in parallel or at separate times. The decoded and/or HTML-stripped data may then be aggregated by the decoding module 208 to generate the plain text 210. The plain text 210 may then be used for further processing.

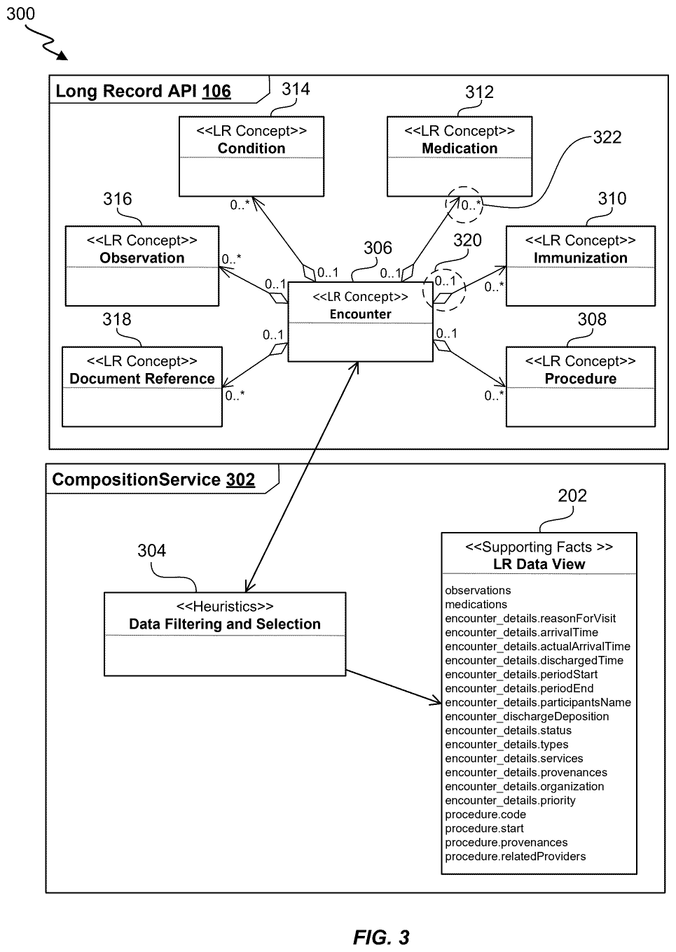

FIG. 3 illustrates an exemplary unified modeling language (UML) diagram 300 of field-value clustering based on association with a specific encounter to generate long record data 202 in accordance with some aspects of the present disclosure. Raw data or unstructured data, as stored in binary files, may comprise of discrete data fields or values that may not have clear relationships between them. The field-value clustering may help in associating such discrete data fields (e.g., medication, condition, procedure, observations, etc.) with a specific event (e.g., an encounter or a procedure), even if those associations may not be specified in the binary files.

A composition service 302 may be responsible for refining and organizing raw, unstructured portions of the EHR data corresponding to a subject. The composition service 302 may work in conjunction with the long record API 106, which may manage the linking and association of discrete data fields with specific encounters. While the long record API 106 may perform task of linking and associating various data fields, the composition service 302 may focus on applying heuristics, filtering, and selecting relevant data fields to present a more organized and meaningful view of the subject's health information.

Heuristics 304 may filter and select the unstructured portions or raw data based on predefined rules or patterns. In some aspects, the heuristics 304 may be based on the filtering criteria, as disclosed herein. Based on the filtering criteria, irrelevant or redundant information may be removed, ensuring that only pertinent data may be included. Data selection further ensures that only the necessary data fields (e.g., clinical observations, medications, and procedures) may be retained, avoiding information overload and noise.

The data fields may then be passed to the long record API 106, which may serve as a specialized interface designed to work with long record (LR) concepts. The long record interface may facilitate the organization and clustering of data fields according to specific encounters. Within the long record API 106, the encounter 306 may serve as a central entity, linking various data fields with subject interactions. The linked data fields may include (for example) immunization 310 (e.g., vaccinations received during the encounter), medications 312 (e.g., prescribed medications associated with the encounter), condition 314 (e.g., diagnosed conditions, allergies, chronic conditions, or any medical conditions noted during the encounter), observations 316 (e.g., vital signs or diagnostic findings), procedure 308 (e.g., surgeries, tests, or diagnostic procedures performed during the encounter), document reference 318 (e.g., lab results, discharge summaries, consultation notes, or imaging reports) etc.

The data fields may be connected to the encounter 306 using UML notations, which may visually represent the relationships between different concepts within the long record API 106. The UML notations, specifically multiplicity indicators 320 and 322, may define the number of instances that may exist between two related data fields in a given relationship. The multiplicity indicator 320 (e.g., 0 . . . 1) may be referred to as zero to one, indicating that the relationship may have zero or one instance of the related encounter. For instance, a condition 314 may typically be associated with only one encounter (e.g., the encounter 306), which means a condition 314 may either be linked to a specific encounter or not at all. The multiplicity indicator 322 (e.g., 0 . . . *) may be referred to as zero to many or many, indicating that the relationship may have zero or more instances of the related encounter. For instance, an encounter 306 may be associated with zero or more conditions (e.g., there may be no conditions or multiple conditions linked to the encounter 306). In some aspects, techniques including field overlap, correlation assessment or other analytical techniques may be employed to identify relationships between seemingly unassociated data fields. For instance, an encounter may include multiple fields such as medications and procedures that are indirectly linked through the clinical context, even though they may not be explicitly tied together in the one or more unstructured portions.

After the data fields have been processed and clustered, the long record data 202 may be presented that may include a variety of semi-structured data elements such as encounter details including the reason for visit (e.g., the primary reason for the subject's visit), arrival time (e.g., the time the subject arrived at the healthcare facility), actual arrival time (e.g., the actual time of arrival, if different from the expected time), discharge time (e.g., the time the subject may be discharged), period start/end (e.g., the start and end dates of the encounter or care period), participants name (e.g., names of the users or care managers required in the encounter), status (e.g., the status of the encounter such as completed or pending), types (e.g., type of healthcare services provided such as outpatient or inpatient), priority (e.g., the urgency of the encounter such as routine or emergency) etc. Additionally, procedure details may include data fields such as code (e.g., procedure code such as ICD-10 code), start time (e.g., the time the procedure may be initiated), and related providers (e.g., information about users who performed or may be involved in the procedure).

The long record data view may be a tabular representation of the semi-structured long record supporting facts, which may not conform to a strict schema, but still comprise of some level of organization including tags or key-value pairs. In one aspect, the long record data 202 may be in a JSON format. In some other aspects, the long record data 202 may be in an xlsx (e.g., excel spreadsheet) format that may include binary files or binary data being stored in cells, enabling easier manipulation and analysis of large datasets or complex relationships between the data elements. The long record data 202 may comprise of any other format including xml, csv, or the like. However, the format of the long record data 202 is not intended to limit the scope of the disclosure.

The long record data 202 may be used during experimentation or inference. In some aspects, the long record data 202 may be used to select relevant data bound to produce a structured output, which may include visit rationales and follow-up instructions. The long record data 202 may be compatible with both offline experimentation and online inference, ensuring flexibility in how the data may be utilized across various stages of data processing and analysis. According to some aspects of the present disclosure, the long record data 202 may be queried and/or decoded, as illustrated in FIG. 2, before being transformed into a structured output using one or more GenAI models. In some other aspects, the long record data 202 may be used by one or more GenAI models to generate a structured output. In some aspects, the long record data 202 may be displayed or output as supporting facts, which may form a part of the structured output.

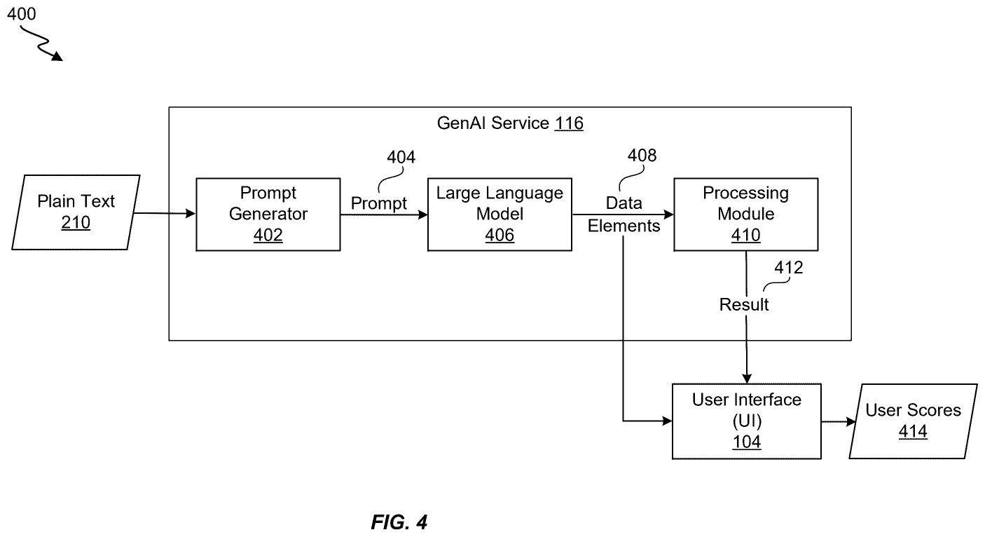

FIG. 4 illustrates an exemplary implementation 400 of transforming the plain text 210 into a result 412 using the one or more GenAI models in accordance with some aspects of the present disclosure. The plain text 210, present in a text-readable format after decoding and HTML-stripping processes, may be input into the GenAI service 116. The GenAI service 116 may have access to one or more GenAI models that may generate a result 412 based on the plain text 210. The one or more GenAI models may comprise of a prompt generator 402, a large language model (LLM) 406 and a processing module 410.

The prompt generator 402 may be employed to generate a prompt 404 based on the plain text 210. The prompt generator 402 may apply prompting techniques including chain of thought (Cot), personification, structured output, reflection, separation of concerns or a combination thereof. Such techniques may ensure that the prompt 404 effectively captures the context of the plain text 210 in a manner that facilitates the accurate extraction of relevant information using the LLM 406.

In some aspects, the prompt generator 402 may incorporate an ontology (e.g., a medical ontology) to efficiently represent the context of the plain text 210 and ensure that the prompt 404 may align with the domain-specific language required for accurate extraction. In some aspects, the ontology may be seen as an automated detection mechanism. The prompt generator 402 may be equipped with knowledge from the ontology, which may automatically identify key concepts including medical terms, procedures, or conditions mentioned in the plain text 210. The ontology may include hierarchical definitions of key concepts including diseases, symptoms, treatments, medications, and body parts, along with their interrelationships (e.g., “is a type of,” “associated with,” or “treated by”). By integrating such structured knowledge, the prompt generator 402 may enable the LLM 406 to process the plain text 210 effectively, ensuring that medical terms and concepts may correctly be understood and represented. The ontology may significantly reduce the likelihood of irrelevant responses, also known as “hallucinations,” or over-summarization by narrowing the context to the pertinent information. The ontology may facilitate generating a prompt 404 with a larger amount of information (e.g., with part or all of the plain text 210).

The prompt 404 may then be processed by the LLM 406 to generate data elements 408 based on the plain text 210. In some aspects, the LLM 406 may utilize models such as Cohere Command R to automatically extract the data elements 408. Cohere Command R may be an instruction-following conversational model that excels at language tasks and offers a longer context window than traditional models. In some aspects, the data elements 408 extracted by the LLM 406 comprise visit rationales (e.g., reason for visit) or follow-up instructions. The data elements 408 may further include, but are not limited to, medical history of a subject, allergy information, diagnoses, symptoms, vital signs, encounter details, procedural details, or clinician's notes. In some aspects, the data elements 408 may be extracted synchronously to reduce latency of the LLM 406.

The result 412 may be generated using few-shot learning, where the prompt 404 may instruct the LLM 406 to extract data elements 408 (e.g., including the visit rationales or chief complaint) corresponding to the plain text 210 using synthetic examples of extracted visit rationales. For instance, examples for visit rationales may include presenting with symptoms (e.g., severe abdominal pain), follow-up care for a diagnosis or medical condition (e.g., diabetes management), addressing specific concerns (e.g., needing a medication refill for asthma), routine or preventive care (e.g., an annual physical examination), referrals or consultations (e.g., an orthopedic evaluation), discussions related to procedures or surgeries (e.g., a preoperative assessment for knee surgery), or medication management (e.g., adjustments to insulin dosage). Thus, the examples for visit rationales or summary examples may guide the LLM 406 about how to process the plain text 210.

The prompt and/or summary examples may also be defined based on a particular use case, type of user, etc. For instance, the LLM 406 may require different guidance in extracting a visit rationale for a follow-up appointment, an emergency hospital visit, or a pediatric appointment. The prompt and/or summary examples may be selected after determining the nature of the visit, either by a qualified user via the UI 104 or automated tools, (e.g., a second LLM) that examine the plain text 210 or the electronic health records.

In some aspects, a single prompt may be used to extract the data elements 408 (e.g., visit rationales or follow-up instructions). In some other aspects, extractions may be performed multiple times (e.g., in parallel or at different time points), using different prompts that may request the extraction of distinct types of information in the form of data elements 408. LangChain may be employed to build and augment the one or more GenAI models, facilitating prompt engineering, and integrating the LLM 406 with relevant data sources or interfaces (e.g., those associated with user).

The processing module 410 may generate the result 412 based on the data elements 408. Upon receiving the outputs from the LLM 406, the processing module 410 may process the data elements 408 to organize and structure it in a predefined format based on a set of transformation criteria. The set of transformation criteria may convert raw information into clearly defined fields, values, or sections that facilitate easier analysis or integration into subsequent workflows. In one aspect, the processing module 410 may apply deduplication to eliminate redundant data, ensuring that only unique, relevant data may be included in the result 412. In some aspects, the processing module 410 may process the data elements 408 and strategically reduce the content to generate the structured output based on the template. The processing module 410 may prepare the subject profile, including key details such as subject demographics, active conditions, and allergies, to be incorporated into the structured output. The processing module 410 may apply filtering to remove irrelevant or low-confidence data, ensuring quality, while also applying error correction to address misclassifications or missing details within the result 412. Normalization may be used to align data with predefined coding systems (e.g., ICD-10, LOINC). In time-sensitive medical situations, the processing module 410 may prioritize the relevant data elements to ensure that their importance may be emphasized.

The result 412 may comprise a structured output (e.g., a summary) including a combination of textual content, numeric data, or other relevant information that may have been systematically arranged in accordance with the intended use case. The summary generated may follow a standardized template that includes one or more of: a timestamp (e.g., current date formatted as month day, year and time when the summary may be generated by the user), followed by the subject's profile with key details (e.g., name, age, sex, active conditions, allergies, etc.), care team, an overview of the last major encounter or successful communication (which may include encounter dates, encounter type location of encounter, responsible provider, prescribed medications for treatment, visit rationale, relevant or abnormal vitals or lab results, procedures, etc.), social factors influencing care (e.g., access to transportation, family circumstances, financial resources) or details on future appointments (e.g., visit rationale, responsible provider, appointment date, RPM readings, etc.). Major encounters, as referred to herein, may include the following encounter types: preregistration, outpatient, inpatient, emergency, urgent care, or telehealth. A “successful communication” may be referred to herein as a case discussion that pertains to the last documented interaction or encounter with the subject, which may be a completed phone call, or any other communication. Any incomplete attempts (e.g., no answer, line busy, or wrong phone number) may not be considered as a part of the result 412.

According to some aspects of the present disclosure, a scoring rubric may be implemented within the processing module 410. The scoring rubric may evaluate the summary's compliance with predefined criteria including accuracy, word count, and relevance, ensuring that the summary may meet necessary standards for healthcare workflows. The scoring rubric may assess the summary based on the following requirements including: the summary may be created using only information available in the EHR data of the subject and may not include any information not captured in that record data, the summary may be within a specified word count range (e.g., between x and y words) and may include time-bound events from the last successful communication event up until the present, the summary may provide a high-level overview of the health record of the subject over the last 12 months (or any other look-back period that may be defined), the summary may not include any recommendations that are inappropriate or irrelevant (e.g., prescribing medications) unless the medications may be specifically relevant to the current condition of the subject etc. Based on the scoring rubric, the structured output or the summary may be enhanced for practical use, which may ensure accuracy and relevance in the healthcare settings.

The summary corresponding to the result 412 may be displayed on the UI 104. Further, the summary may comprise a segment of the long record data 202 or the EHR data derived from the EHR 108 that may be structured. The segment may comprise of supporting facts that may provide a detailed description of a subject's overall condition and known allergies, along with dates associated with key events or diagnoses. In some aspects, the supporting facts may complement the summary by offering supporting details, adding clarity to the summarized information. In some other aspects, supporting facts may provide more granular data that further enriches the summary and may further be utilized for decision-making or clinical review. The generated summary may comprise a link which, when selected, directs the user to a supporting facts page. The link may serve as a gateway to more detailed information that supports the summary displayed on the UI 104.

Some aspects relate to the result 412 that may be other than the structured output (e.g., the summary). Other results may include talking points (e.g., scripted prompts for care managers who prefer phone calls), call activity logs (e.g., calls made, call summaries, interactions with the patient), care plan updates (e.g., modifications to treatment plans, setting of health goals), reminders (e.g., scheduled tasks, follow-ups for users), longitudinal plan (e.g., goals and interventions, personalized for each subject), longitudinal record (e.g., customized summaries for defined look-back periods, tailored to specific care programs), outreach messages (e.g., personalized communication for enrolling subjects in care management programs), care handoff documentation (e.g., transition documents for pediatric subjects aging into adult care) or a combination thereof.

In some aspects, the result 412 displayed on the UI 104 may be subject to user feedback, with users providing a score (herein referred to as user scores 414) to guide further refinements of the one or more GenAI models. Users may score the result 412 using interactive elements including approval or disapproval icons (e.g., thumbs-up or thumbs-down), editable text fields, or a numerical scoring system to express their satisfaction with the result 412. If a result 412 receives a low score—based on user feedback, such as a threshold percentage of unsatisfactory ratings—the system may refine the prompt and/or summary examples used to guide the one or more GenAI model's output. In some aspects, part, or all of the result 412 may be provided to the subject after assigning the user scores 414. In some aspects, the user may score the data elements 408 in addition to the result 412. The feedback may enable the GenAI service 116 to continuously improve, ensuring that subsequent extractions and/or summaries may be more aligned with user expectations and better suited to the needs of the healthcare environment.

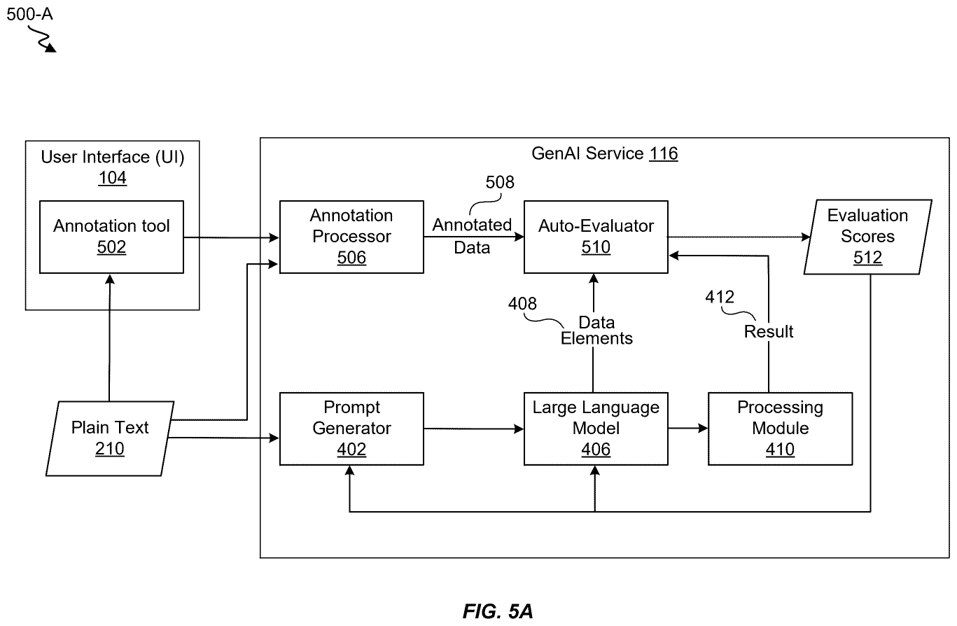

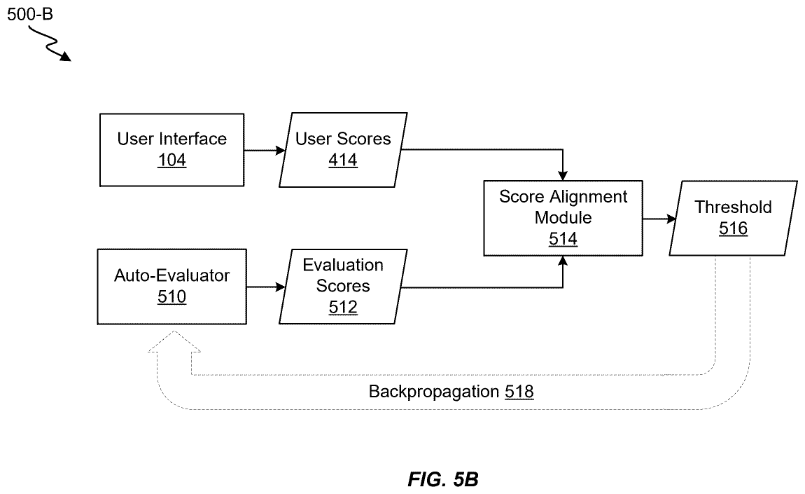

FIG. 5A illustrates an exemplary process 500-A for evaluating and training the one or more GenAI models based on evaluation scores generated by an auto-evaluator 510 in accordance with some aspects of the present disclosure. A GenAI model, more specifically another LLM (herein referred to as the auto-evaluator 510) with configurations or even architecture that may be different from the LLM 406 used for extracting data elements 408—may automatically generate an evaluation scores 512 predicting a quality of the data elements 408 or the result 412. Exemplary frameworks that may be used include Langchain (for example) ChatOCIGenAI for interfacing with the LLM 406 and LangFuse to automatically evaluate the quality of the data elements 408 or the result 412. For instance, LangFuse may evaluate and monitor their quality by comparing them with predefined evaluation criteria or user input. The evaluation scores 512 may be generated by comparing part or all of output generated using an LLM 406 (e.g., data elements 408) and the processing module 410 (e.g., a summary, talking points, etc.) with those generated by or with augmentation with the user input (e.g., annotated data 508).

An annotation tool 502 may be used to identify annotations 504 by utilizing input from user annotators, who follow defined labeling guidelines to label the plain text 210. In some aspects, the annotation tool 502 may be integrated into or functionally part of the UI 104. An annotation tool 502 may be any software application that enables user annotators to label and categorize elements within a given dataset (e.g., the plain text 210). According to some aspects of the present disclosure, the annotation tool 502 may be ART, a tool that enables user annotators to interact with and annotate the plain text 210. Other annotation tools may include BRAT, Prodigy, Labelbox, Doccano etc. The annotations 504 provided by the annotation tool 502 may comprise documents typically in JSON format including string indices. In some aspects, the annotations 504 may include documents or metadata that provide a structured representation of the labels and their corresponding locations within the plain text 210.

Each annotation in the JSON document includes both the label assigned to a particular element (such as a medical term, procedure, or condition) and the precise location of that element within the plain text 210. Such annotations may function as a map or guide to highlight important pieces of information in the raw plain text. For instance, in the text “John Doe, a 45-year-old male with hypertension, visited the clinic on Jan. 12, 2025, complaining of chest pain and shortness of breath,” the label “patient name” or “subject name” is assigned to “John Doe” and is located at positions 0-8 in the plain text. Similarly, the label “age” is assigned to “45-year-old” with a location at positions 10-18, “hypertension” is labeled as “condition” and appears at positions 45-56, “visit date” label corresponds to “Jan. 12, 2025” and spans from position 58 to 76, “chestpain and shortness of breath” are labeled as “symptoms” and are found at positions 77-111.

An annotation processor 506 may compare the annotations 504 with the plain text 210. The annotation processor 506 may evaluate the annotations 504 in relation to the plain text 210 and enrich them with additional context. The enrichment may include supplementing the annotations 504 with extra information such as clarifying details or linking the annotated elements to relevant data points within the plain text 210. Enrichment may be done by using techniques such as natural language processing (NLP), named entity recognition (NER), relation extraction, context-based reasoning, semantic analysis etc. Based on the enrichment, a separate JSON object may be generated that may serve as the annotated data 508. The annotated data 508 may provide a comprehensive understanding of the plain text 210 and further be used for model training or detailed analysis.

The auto-evaluator 510 may compare the annotated data 508 with the data elements 408 and the result 412 to generate evaluation scores 512. The comparison assesses how well the generated outputs (e.g., the data elements 408 and the result 412) aligns with the annotated data 508, considering accuracy, relevance, and completeness. In some aspects, the evaluation scores 512 may be represented as a single aggregated score assigned to the data elements 408 or the result 412. In some other aspects, the evaluation scores 512 may be assigned on a per-element basis, where each individual data element of the data elements 408 or section of the result 412 receives its own evaluation score. Such scores may reflect how well each part of the output aligns with the annotated data 508.

Based on the evaluation scores 512, the one or more GenAI models including the LLM 406 and the prompt generator 402 may be trained or fine-tuned. In some aspects, the generated evaluation scores 512 may be used in conjunction with a loss function or objective function to facilitate the training of the one or more GenAI models. The objective function may be represented as: J(θ)=max_{θ}(α·Accuracy(yᵢ,ŷᵢ)−β·L(θ, S)), where α and β are hyperparameters controlling the importance of accuracy and evaluation scores, Accuracy(yᵢ,ŷᵢ) measures how closely the predicted output matches the true labels and L(θ, S) represents the loss function, which may be modified using the evaluation scores S. The objective function may balance the trade-off between accuracy and the evaluation scores 512, ensuring that the one or more GenAI models may be trained to raise overall performance on both fronts.

Alternatively, a loss function may adjust the weight of each data element's loss based on its evaluation score, ensuring that higher-quality predictions may be given less penalty (lower loss), while lower-quality predictions may be penalized more. The loss function may be represented as:

$$L ( θ , S ) = \frac{1}{N} {\sum }_{i = 1}^{N} {S}_{i} \cdot Loss ( {y}_{i} , {\hat{y}}_{i} ) ,$$

where N is the number of data elements present in the plain text 210, Sᵢ is the evaluation score for the ith data element, yᵢ is the true value (e.g., annotated data) and ŷ₁ is the predicted output by the LLM 406. Then, Gradient descent may be used to minimize the loss function by iteratively adjusting the parameters of the LLM 406 or the prompt generator 402, represented as: θₜ₊₁=θₜ·η∇_{θ}L(θ, S), where θₜ may be the models parameter at iteration t, η is the learning rate and ∇_{θ}L(θ, S) is the gradient of the loss function with respect to the one or more GenAI models parameters.

FIG. 5B illustrates an exemplary process 500-B for training the auto-evaluator 510 based on a threshold 516 in accordance with some aspects of the present disclosure. Training of the auto-evaluator 510 may be facilitated by a score alignment module 514 that compares the evaluation scores 512 generated by the auto-evaluator 510 with the user scores 414 assigned by the user via the UI 104. The user scores 414 may serve as a ground truth, and the comparison process assesses how closely the outputs of the LLM (e.g., the auto-evaluator 510) align with the user evaluations. The comparison may include calculating a similarity metric or error using mean square error (MSE) or mean absolute error (MAE), which may be represented as:

$$M S E = \frac{1}{N} {\sum }_{i = 1}^{N} {( E {S}_{i} - U {S}_{i} )}^{2} ,$$

where ESᵢ represents the evaluation scores 512, USᵢ represents the user scores 414 and N represents the number of data elements.

Based on the comparison, the score alignment module 514 calculates a threshold 516, which represents the difference (or lack of alignment) between the auto-evaluator's scores and the user's scores. The threshold 516 may be calculated as a performance metric such as the error margin or a confidence level. For instance, if the MSE or MAE may be below a certain value, the score alignment module 514 may indicate that the auto-evaluator 510 has achieved acceptable performance. Alternatively, the threshold 516 may represent the percentage of data elements for which the auto-evaluator's score is within a predefined margin of error from the user scores 414. The threshold 516 may be calculated as: Theshold=performance metric (e.g., MSE, MAE)<ϵ, where ϵ may be a small error margin threshold that determines when the auto-evaluator 510 may be performing adequately.

Once the threshold 516 may be calculated, the threshold 516 may be fed back into the auto-evaluator 510 through backpropagation 518, which may be used to adjust the parameters of the auto-evaluator 510 to reduce discrepancies between the evaluation scores 512 and the user scores 414. Such iterative process may improve the auto-evaluator's ability to predict user scores 414 by fine-tuning its underlying model. In some aspects, the threshold 516 may be backpropagated until the auto-evaluator 510 reaches a high degree of confidence, often represented by a confidence threshold (e.g., 90% or higher), indicating that the model or the auto-evaluator 510 may reliably be producing evaluation scores 512 that closely match user assessments. Once the confidence threshold may be achieved, the auto-evaluator 510 may automatically manage rapid iteration and validation using the evaluation scores 512, as illustrated in FIG. 5A.

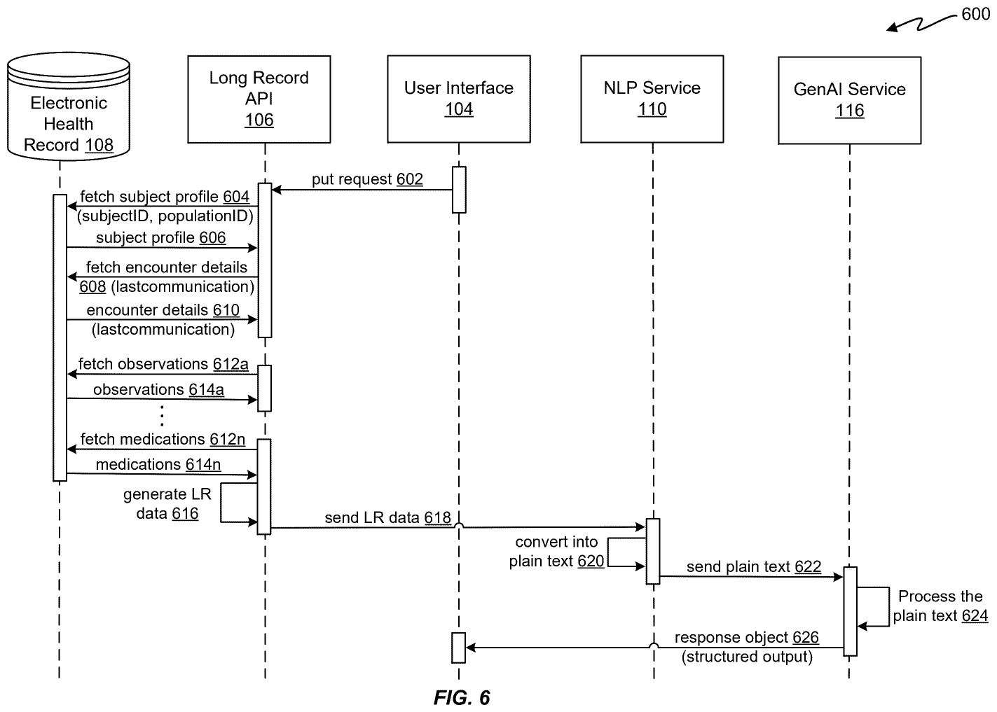

FIG. 6 illustrates an exemplary sequence diagram 600 for extracting and structuring one or more unstructured portions into a result 412 using business logic and the one or more GenAI models in accordance with some aspects of the present disclosure. Business logic may be part of a software that defines the operations, calculations or data processing logic based on the requirements of the business or system. The workflow, including operations to retrieve, process and transform the EHR data into a plain text 210, may be part of the business logic that ensures the proper handling of data to meet healthcare objectives (e.g., generating summaries for the users).

At 602, a user may put a request to generate a result, specifically a structured output, via the UI 104. The request may comprise of identifiers corresponding to a subject including (for example) subject ID, population ID or encounter date. The encounter date may correspond to a reference date to the recent communication event or last communication event. In some aspect, the subject may have had one or more encounters after the last communication event (e.g., with different healthcare providers). Based on the request, the long record API 106 may retrieve the one or more unstructured portions from the EHR 108 or the database.

At 604, the long record API 106 may fetch subject profile based on the subject ID and the population ID. At 606, the EHR 108, in response, may send the subject profile to the long record API 106. The subject profile include (for example) name, age, sex, conditions (e.g., diagnosis and symptoms) or allergies. At 608, the long record API 106 may fetch encounter details from the EHR 108 based on the encounter date. The encounter details may be retrieved by the EHR 108, at 610. If encounter date is specified, the one or more unstructured portions associated with the number of encounters that may have occurred up until the recent communication event may be retrieved. In some instances, if the encounter date is not specified, the long record API 106 (by default) may retrieve the one or more unstructured portions associated with encounters from a specific look-back period. In some aspects, the look-back period may be 3 months. In some aspects, the look-back period may be other than 3 months.

In some aspects, the long record API may fetch additional information (e.g., relevant data elements within the one or more unstructured portions) associated with the encounters from the EHR 108 or the database based on a request (e.g., a request to generate a summary). One or more API calls may be made to fetch the additional information including (for example) observations, medications, procedures, immunizations etc. At 612a, the long record API 106 makes a call to fetch observations associated with an encounter. The observations including (for example) vital signs or diagnostic findings may be retrieved from the EHR 108, at 614a. At 612n, the long record API 106 may fetch medications associated with the one or more encounters. The medications including (for example) prescribed medications associated with the one or more encounters may be retrieved from the EHR 108, at 614n. In between 612a-n, multiple other API calls may be made to retrieve the other one or more unstructured portions associated with the one or more encounters.

After retrieving the one or more unstructured portions, the long record API 106 may aggregate the portions. At 616, the long record API 106 may associate discrete data elements within one or more unstructured portions, which may otherwise remain unconnected. For instance, the long record API 106 may link the observations, medications, conditions etc. (using part or all of the data elements retrieved at s 614a-n) with an encounter of the one or more encounters that may be retrieved in part at 610 to generate the long record data 202. The long record data 202 may be a tabular representation of the semi-structured long record supporting facts that may comprise of some level of organization. In one aspect, the long record data 202 may be in a JSON format. In some other aspects, the long record data 202 may be in an xlsx format. However, the format of the long record data 202 is not intended to limit the scope of the disclosure.

At 618, the long record data 202 may be sent to the NLP service 110, where the semi-structured data may be transformed into a text-based format (e.g., a plain text). At 620, the decoding module 208 present within the NLP service 110, may decode and/or strip the long record data 202, as illustrated in FIG. 2. After decoding, the long record data 202 may be converted into a plain text 210.

At 622, the plain text 210 may be sent over to the GenAI service 116. At 624, the GenAI service may access the one or more GenAI models to process the plain text 210 and transform the plain text 210 into a structured output (e.g., a summary). The one or more GenAI models may generate a prompt 404 based on the plain text 210, extract data elements 408 using an LLM 406 and process the data elements 408 to generate a structured output. The structured output may be a JSON object. In some aspects, the structured output may be in a rich text format (rtf) format. The one or more GenAI models may generate the summary in a specific language (e.g., english) to ensure that the content may be understandable and accessible for the intended users. In some aspects, the summary may be generated in other languages, depending on user preferences or regional requirements, ensuring global accessibility and relevance. Summarization latency may be reduced to be less than 1 minute, enabling rapid generation of summaries for timely decision-making. At 626, the response object (e.g., the summary) may be rendered on the UI 104. The user may review the summary or the structured output via the UI 104.

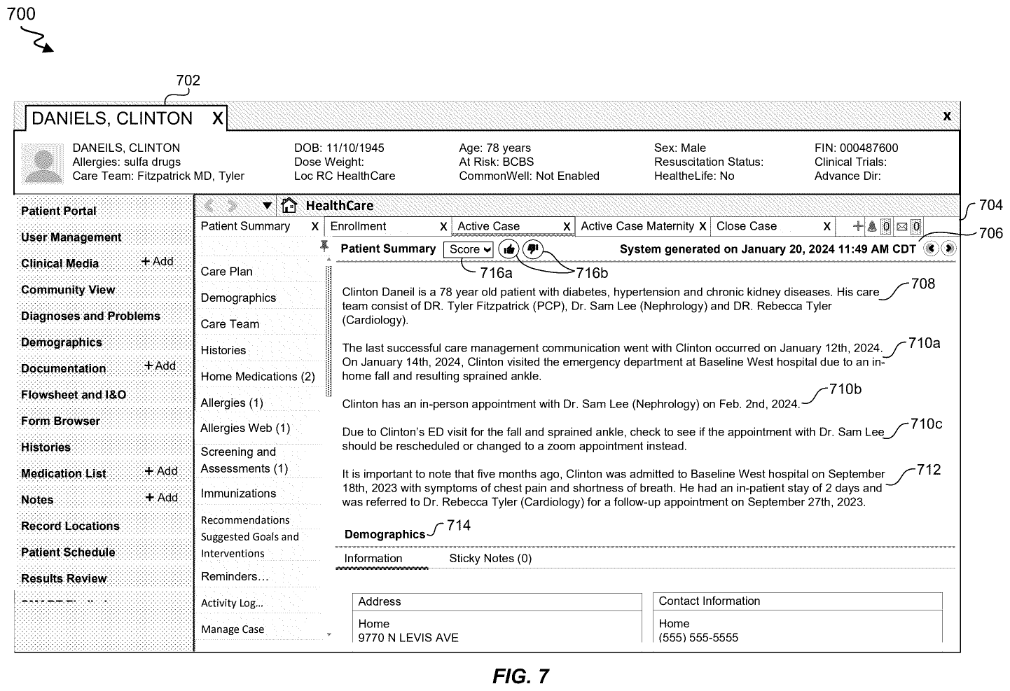

FIG. 7 illustrates an exemplary healthcare portal 700 of a subject within a healthcare management platform in accordance with some aspects of the present disclosure. The exemplary healthcare portal 700 may incorporate a summary generated by the one or more GenAI model, which may serve as a valuable tool for the users. The summary may provide an overview of the health status of a subject, enabling users to identify key changes, follow-up points, and necessary adjustments to the care plan, which may ultimately guide informed decision-making and treatment recommendations for the subject.

The summary, as illustrated in the exemplary healthcare portal 700 of a subject, may be generated based on the standardized template. The portal may present the subject profile 702, which includes key details such as name, date of birth (DOB), age, sex, active conditions (with a specific lookback period), allergies, care team, financial identification number (FIN), resuscitation status, clinical trials or advance directives (if available), risk of a condition, Common Well status etc. Within the portal, a well-structured navigation tab 704 may provide the user with access to the subject's summary, enrollment status, active case, active maternity case, closed case, etc. The “active case” section may present the summary of the subject along with a timestamp 706 (e.g., the date and time when the summary may be generated by the user). The summary may comprise of one or more sections including a subject overview 708, time-bound encounters 710a-c and longitudinal summary 712.

The subject overview 708 may include demographic and health information about the subject, which may include their name, age, sex, active conditions, allergies, and care team members. In some aspects, the subject overview 708 may provide a concise snapshot of the subject's health profile, highlighting critical information relevant to ongoing care and treatment planning. For instance, the subject may be a 78-year-old patient with diabetes, hypertension, and chronic kidney disease, with a care team consisting of specialists such as a primary care provider (PCP), nephrologist, and cardiologist.

The time-bound encounters 710a-c may outline the recent and upcoming encounters associated with the subject that may focus on events including care management communications, emergency visits or scheduled appointments. For instance, time-bound encounters 710a-c may include details of the last successful communication with the user (e.g., care manager or care management team), a visit to the emergency department due to an injury, and any upcoming in-person or virtual appointments. Additionally, the time-bound encounter 710a-c may flag any necessary follow-up actions (e.g., rescheduling appointments or adjusting the method of consultation) based on recent health events.

The longitudinal summary 712 may provide an overview of the subject's health records and social determinants of health (SDOH) over a defined look-back period (e.g., a 3-month, 6-month, or 12-month look-back period). The longitudinal summary 712 may highlight critical events including hospital admissions, past treatments, changes in the health status of the subject etc. In some aspects, the longitudinal summary 712 may also reflect important social factors (e.g., access to care, financial status, and living conditions) that may impact the subject's health outcomes. For instance, if a subject's family may have experienced financial hardship (e.g., being unable to afford food, utilities, or clothing) that may be reflected in the summary, highlighting potential barriers to care or adherence to the treatment.

Additionally, the summary may be scored by the user through interactive elements 716a-b. A score box 716a may enable the user to enter a numerical score directly, or alternatively, select a score from a drop-down menu. Additionally, icons 716b (e.g., thumbs-up and thumbs-down) may enable the user to express their satisfaction with the result 412. Such interactive scoring options may enable the user to quickly and easily evaluate the quality of the result 412, providing valuable feedback to enhance the overall healthcare portal experience.

The exemplary healthcare portal 700 of the subject may also display demographics 714 corresponding to the subject, which may include information such as address and contact information of the subject. The sticky notes feature within the demographics 714 of the exemplary healthcare portal 700 may enable the user to add personalized, informal annotations or reminders related to the subject's demographic details. In some aspects, the sticky notes may provide a flexible and accessible way for users to capture quick, important insights that may not fit within the structured data fields but still play a role in supporting the subject's care.

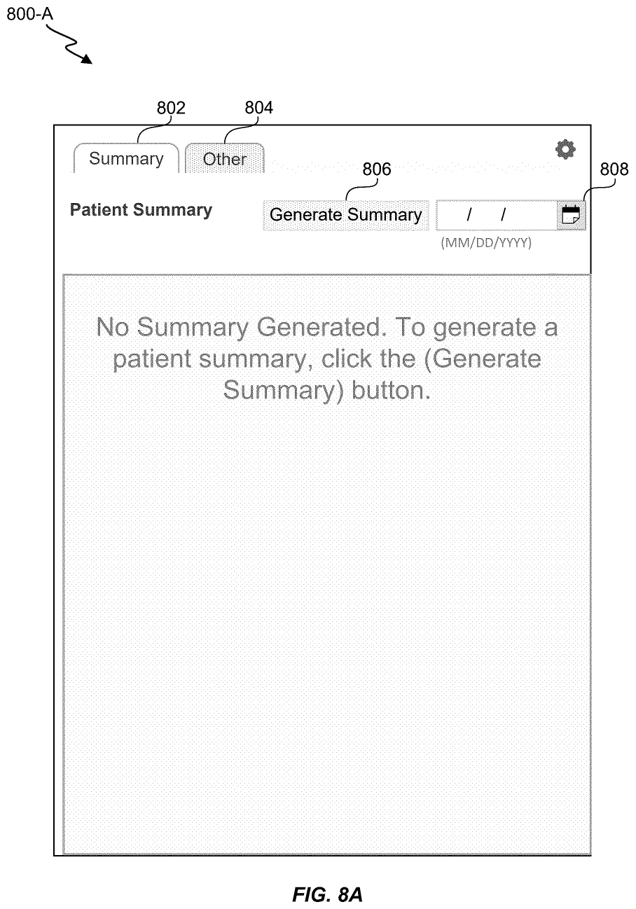

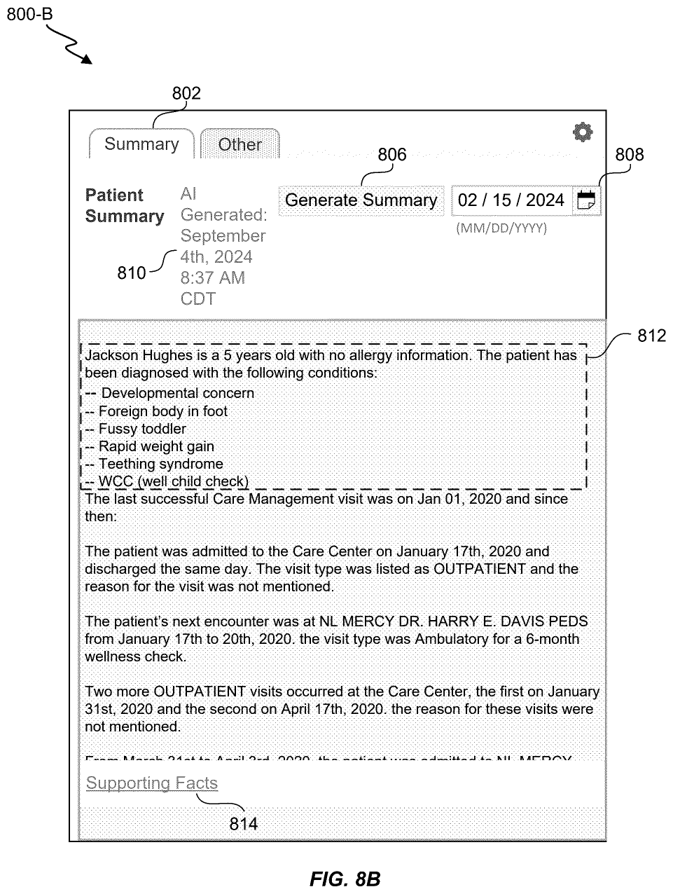

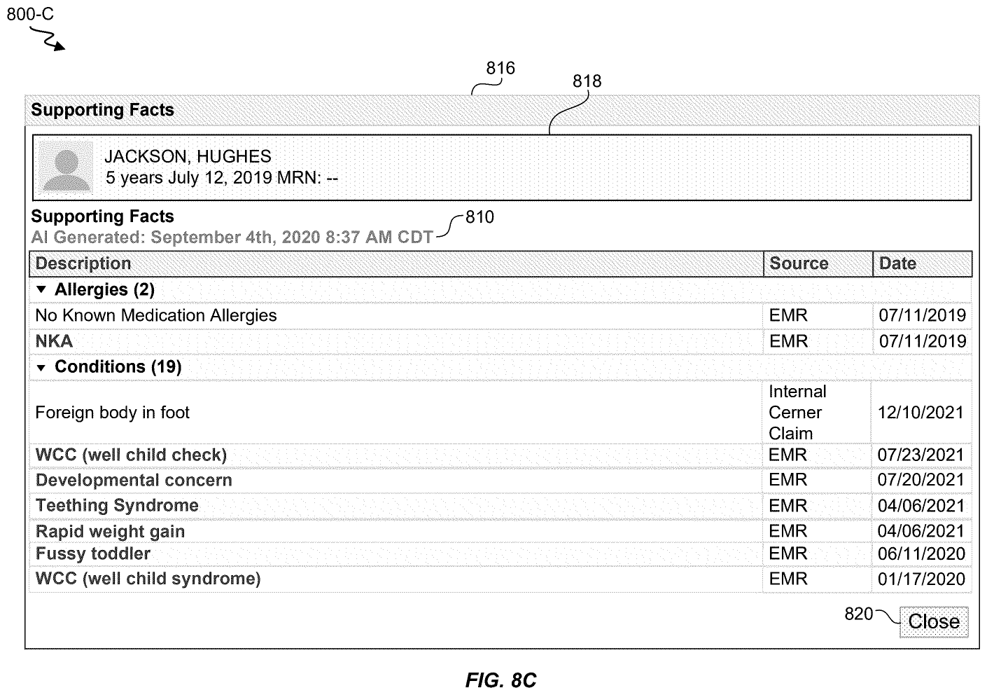

FIG. 8A illustrates an exemplary user interface 800-A that the user may experience when accessing a summary prior to its generation within the healthcare management platform, in accordance with some aspects of the present disclosure. The exemplary user interface 800-A may appear as a sidebar within the existing workflow in the UI 104 or healthcare management platform. A summary tab 802, illustrated within the exemplary UI 800-A, may serve as the central location where the user may generate and view the summary of a subject. The summary tab 802 may provide access to the summary functionality within the healthcare management platform. In addition to the summary tab 802, other tabs 804 may also provide access to different sections of the healthcare management platform (e.g., appointments, scheduling, medication management etc.).

Within the exemplary user interface 800-A, a date picker 808 may enable the user to select a specific date corresponding to a communication event that may be of interest. The communication event may typically refer to a prior interaction or encounter with the subject that may be a visit, phone call, or other form of communication. By selecting the date via the date picker 808, the user may choose to generate a summary based on the subject's health status as of that specific communication event, which may be useful for tracking progress or reviewing the subject's condition before or after that particular point in time. In some aspects, a look-back range may be incorporated, and once a date may be selected via the date picker 808, the result 412 may be automatically generated based on the communication event data within the specified time-bound range.

Once the user may have selected the relevant date in the date picker 808, a generate summary button 806 may be clicked to generate the summary based on the data associated with that date. The summary will then be displayed, offering insights into the subject's health condition, treatments, and any relevant updates from the communication event selected.

FIG. 8B illustrates an exemplary user interface 800-B featuring a summary generation within the healthcare management platform in accordance with some aspects of the present disclosure. The user may select a communication event date using the date picker 808 within the summary tab 802. Once the date may be selected, the user may generate the summary by clicking the generate summary button 806. The generated summary may include a timestamp 810, which shows the current date and time when the summary may be generated by the one or more GenAI models.

The summary may incorporate a subject overview 812 that includes information such as name, age, allergy details, and diagnosed conditions of the subject. The remainder of the summary may feature time-bound encounters that may have occurred based on the selected communication event date. The summary generated may be based on the set of transformation criteria, as disclosed herein, which may include logic and format requirements. Logic requirements may specify how data may be processed or managed, while format requirements may determine how the data may be presented. For instance, for the subject's name (logic requirement: first and last name, ignore middle name; format requirement: capitalize both first and last name), for age (logic requirement: age in years; format requirement: use numbers), for gender (logic requirement: default to “subject” or “patient” if missing; format requirement: lowercase), and for allergies (logic requirement: show active allergies only; format requirement: comma-separated list).

Similarly, for encounters (logic requirement: show supported types, provide relevant dates, location as hospital/clinic name; format requirement: sentence format, Month DD, YYYY), for medications (logic requirement: link to supported encounters, exclude drugs without dosage; format requirement: display medications with dosage), for labs (logic requirement: show non-normal interpretations; format requirement: bulleted or sentence format), for vital signs (logic requirement: show non-normal interpretations; format requirement: bulleted list with values and units), for procedures (logic requirement: show all procedures without filtration; format requirement: bulleted list), and for conditions (logic requirement: last 5 years, exclude non-medical conditions; format requirement: bulleted list, long form, capitalized).

Further, the users may need to validate the workflow with supporting details by clicking into an event, link, or hyperlink to view additional details the generated summary may provide. By pressing a link 814, users may access a detailed, organized view of the underlying data that contributed to the creation of the summary. Such data (e.g., supporting facts) may include lab results, medication history, clinical notes, and other key health information pulled from the EHR 108. The supporting facts may enable users to verify and review the generated summary, providing transparency and supporting the clinical decisions reflected in the summary.

FIG. 8C illustrates an exemplary segment 800-C of at least a portion of the structured output accessed by the link 814 in accordance with some aspects of the present disclosure. The exemplary segment 800-C illustrates a supporting facts page 816 that may be accessed from the link 814 illustrated in the exemplary UI 800-B. The supporting facts page 816 may refer to the subject by displaying the subject profile 818 and also replicate the timestamp 810 to reflect the date and time when the summary may have been generated. The supporting facts page 816 may present a detailed and organized compilation of information from multiple sources (e.g., the EHR 108 or the database), where the information may include lab results, imaging reports, clinical notes etc. The supporting facts may provide supporting details for the generated summary, including conditions or allergies that may be mentioned in the summary. In some aspects, the supporting facts may only present the abnormal vitals or lab results that may be important to take into consideration. In some other aspects, the supporting facts page 816 may provide a thorough description of the subject's overall health condition, detailing any known allergies, chronic conditions, treatments, and medications.

The supporting facts page 816 may also include relevant sources and dates for each event, diagnosis, or clinical interaction, offering a timeline of the patient's health journey. In some aspects, the supporting facts page 816 may enable the users to review the underlying data that informed the generated summary, ensuring transparency, accuracy, and a comprehensive view of the subject's history. Additionally, the supporting facts may provide context around specific health events including hospital admissions or significant changes in the condition of the subject, to aid in decision-making and care planning.

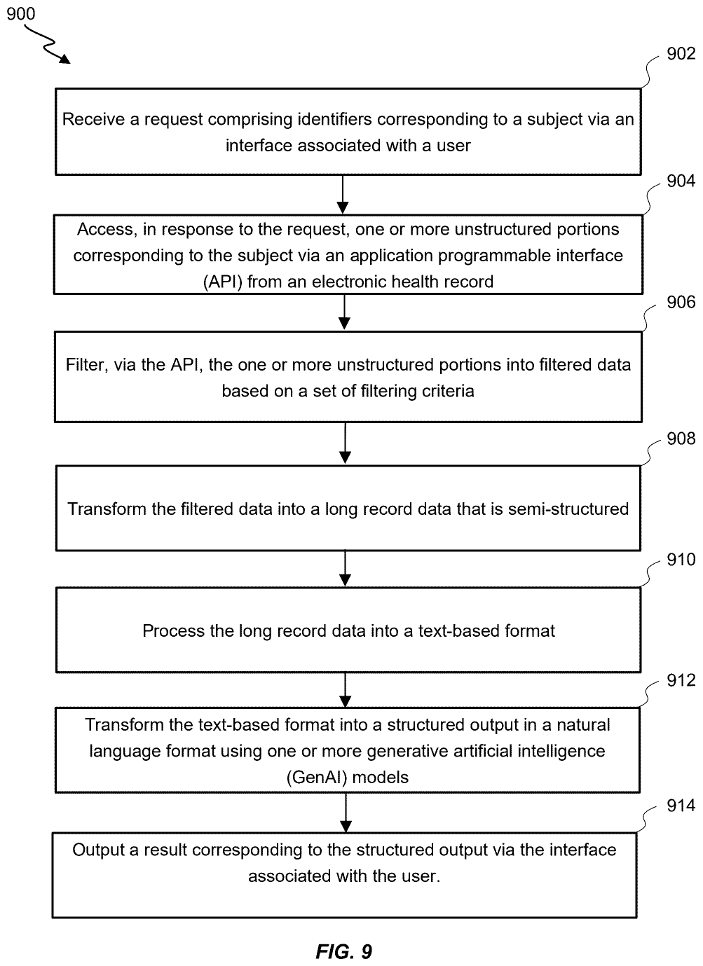

FIG. 9 shows an example flowchart of a system implementing a strategic content reduction to facilitate the user communications in accordance with some aspects of the present disclosure. The blocks in workflow 900 are illustrated in a specific order, while the order can be modified, for example, some blocks may be performed before other, and some blocks may be performed simultaneously. The blocks may be performed by hardware, software, or a combination thereof.

At block 902, a request may be received by the user via the UI 104 to generate a structured output (e.g., a summary) as a result. In some aspects, the request may include generating talking points to assist the user, or providing a summary of recent calls or communication between a user and a subject. In some other aspects, the request may include generating key insights, action items, follow-up tasks, a sentiment analysis of the conversation, or even creating a concise report based on a communication. Further, the request may comprise of identifiers corresponding to a subject, for which a result may be generated. The identifiers may include, but are not limited to, subject ID, population ID, encounter date (e.g., a reference date to a recent communication event or last communication event). The identifiers (e.g., subject ID or population ID) may also be used as an identification to uniquely identify a subject. The identifier (e.g., encounter date) may be used to identify one or more unstructured portions associated with one or more encounters.

At block 904, a long record API 106 may retrieve the subject profile and/or the one or more unstructured portions based on the request. The one or more unstructured portions include binary files comprising raw data. At block 906, the one or more unstructured portions may be filtered by the long record API 106 based on a set of filtering criteria or filtering criteria. The filtering criteria may apply specific logic to exclude unstructured portions including keywords (for example) “form”, “education”, “edu”, “questionnaire”, “referral”, “prescription letter”, or any other “letter” present in the titles of the unstructured portions. In some aspects, the long record API 106 may retrieve the one or more unstructured portions based on the set of filtering criteria, as disclosed in FIG. 6. Based on the filtering criteria, filtered data may be generated.

At block 908, the filtered data may be transformed into a semi-structured format (e.g., long record data 202). The transformation may be performed by the long record API 106 in conjunction with a service (e.g., a composition service 302). The composition service 302 may filter or select the data elements within the filtered data, which may be associated. The long record API 106 may utilize techniques such as field-value clustering to associate data elements with a specific encounter that may otherwise be unassociated. After association, the composition service 302 may generate a long record data view that includes the long record data 202 in (for example) JSON format.

At block 910, the long record data 202 may be processed by the decoding module 208. The decoding module 208 may decode and/or strip the long record data 202 into a plain text 210 (text-based format). For instance, if the long record data 202 may be a JSON object, the decoding module 208 may only strip of tags, scripts, or other non-text elements from the long record data 202. If the long record data 202 may be in xlsx format, the decoding module 208 may decode and strip the data to produce the plain text 210.

At block 912, the plain text 210 or the text-based format may be transformed into a structured output using the one or more GenAI models. The one or more GenAI models may be used to generate a prompt 404 based on the plain text 210, extract data elements 408 using an LLM 406 based on the prompt 404 and process the data elements 408 using a processing module 410 to generate the structured output (e.g., the summary) in natural language format using a set of transformation criteria. In some aspects, the structured output may be in a rtf format that may support text formatting and structure. At block 914, a result corresponding to the structured output may be displayed on the UI 104 associated with the user. The structured output may assist the user by reducing time expenditure and resource usage.

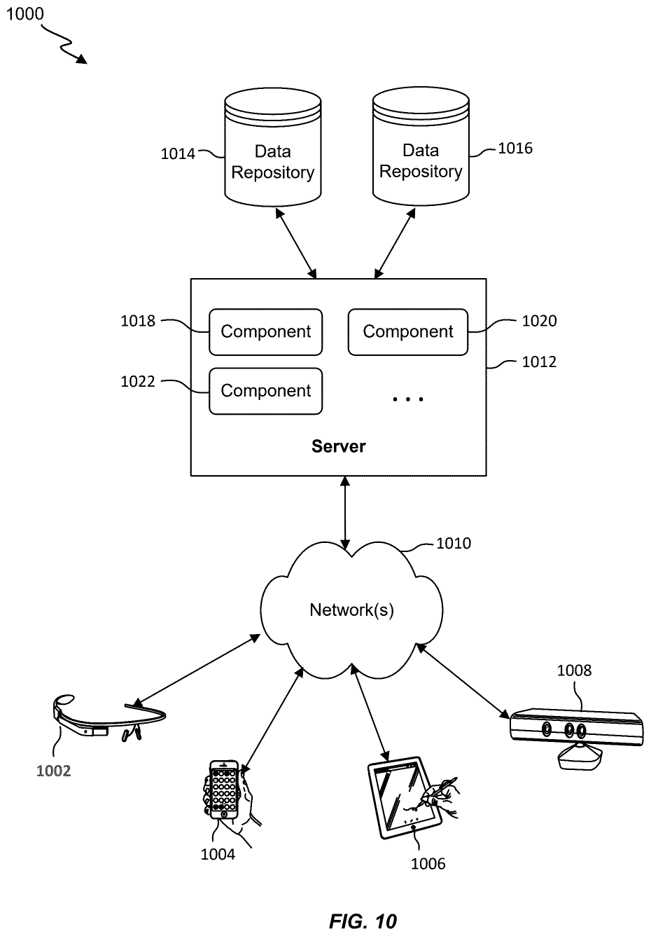

FIG. 10 depicts a simplified diagram of a distributed system 1000 for implementing an aspect in accordance with some aspects of the present disclosure. In the illustrated aspect, distributed system 1000 includes one or more client computing devices 1002, 1004, 1006 and/or 1008 coupled to a server 1012 via one or more communication networks 1010. Clients computing devices 1002, 1004, 1006 and/or 1008 may be configured to execute one or more applications.

In various aspects, server 1012 may be adapted to run one or more services or software applications that enable techniques disclosed herein.

In certain aspects, server 1012 may also provide other services or software applications that can include non-virtual and virtual environments. In some aspects, these services may be offered as web-based or cloud services, such as under a Software as a Service (SaaS) model to the users of client computing devices 1002, 1004, 1006 and/or 1008. Users operating client computing devices 1002, 1004, 1006 and/or 1008 may in turn utilize one or more client applications to interact with server 1012 to utilize the services provided by these components.

In the configuration depicted in FIG. 10, server 1012 may include one or more components 1018, 1020 and 1022 that implement the functions performed by server 1012. These components may include software components that may be executed by one or more processors, hardware components, or combinations thereof. It should be appreciated that various different system configurations are possible, which may be different from distributed system 1000. The aspect shown in FIG. 10 is thus one example of a distributed system for implementing an embodiment system and is not intended to be limiting.

Users may use client computing devices 1002, 1004, 1006 and/or 1008 for techniques disclosed herein. A client device may provide an interface that enables a user of the client device to interact with the client device. The client device may also output information to the user via this interface. Although FIG. 10 depicts only five client computing devices, any number of client computing devices may be supported.

The client devices may include various types of computing systems such as smart phones or other portable handheld devices, general purpose computers such as personal computers and laptops, workstation computers, personal assistant devices, smart watches, smart glasses, or other wearable devices, equipment firmware, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux® or Linux-like operating systems such as Oracle® Linux and Google Chrome® OS) including various mobile operating systems (e.g., Microsoft Windows Mobile®, iOS®, Windows Phone®, Android®, HarmonyOS®, Tizen®, KaiOS®, Sailfish® OS, Ubuntu® Touch, CalyxOS®). Portable handheld devices may include cellular phones, smartphones, (e.g., an iPhone®), tablets (e.g., iPad®), and the like. Virtual personal assistants such as Amazon® Alexa®, Google® Assistant, Microsoft® Cortana®, Apple® Siri®, and others may be implemented on devices with a microphone and/or camera to receive user or environmental inputs, as well as a speaker and/or display to respond to the inputs. Wearable devices may include Apple® Watch, Samsung Galaxy® Watch, Meta Quest®, Ray-Ban® Meta® smart glasses, Snap® Spectacles, and other devices. Gaming systems may include various handheld gaming devices, Internet-enabled gaming devices (e.g., a Microsoft Xbox® gaming console with or without a Kinect® gesture input device, Sony PlayStation® system, Nintendo Switch®, and other devices), and the like. The client devices may be capable of executing various different applications such as various Internet-related apps, communication applications (e.g., e-mail applications, short message service (SMS) applications) and may use various communication protocols.

Network(s) 1010 may be any type of network familiar to those skilled in the art that can support data communications using any of a variety of available protocols, including without limitation TCP/IP (transmission control protocol/Internet protocol), SNA (systems network architecture), IPX (Internet packet exchange), AppleTalk®, and the like. Merely by way of example, Network(s) 1010 can be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Server 1012 may be composed of one or more general purpose computers, specialized server computers (including, by way of example, PC (personal computer) servers, UNIX® servers, LINIX® servers, mid-range servers, mainframe computers, rack-mounted servers, etc.), server farms, server clusters, a Real Application Cluster (RAC), database servers, or any other appropriate arrangement and/or combination. Server 1012 can include one or more virtual machines running virtual operating systems, or other computing architectures involving virtualization such as one or more flexible pools of logical storage devices that can be virtualized to maintain virtual storage devices for the server. In various aspects, server 1012 may be adapted to run one or more services or software applications that provide the functionality described in the foregoing disclosure.

The computing systems in server 1012 may run one or more operating systems including any of those discussed above, as well as any commercially available server operating system. Server 1012 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, SAP®, Amazon®, Sybase®, IBM® (International Business Machines), and the like.

In some implementations, server 1012 may include one or more applications to analyze and consolidate data feeds and/or event updates received from users of client computing devices 1002, 1004, 1006 and/or 1008. As an example, data feeds and/or event updates may include, but are not limited to, blog feeds, Threads® feeds, Twitter® feeds, Facebook® updates or real-time updates received from one or more third party information sources and continuous data streams, which may include real-time events related to sensor data applications, financial tickers, network performance measuring tools (e.g., network monitoring and traffic management applications), clickstream analysis tools, automobile traffic monitoring, and the like. Server 1012 may also include one or more applications to display the data feeds and/or real-time events via one or more display devices of client computing devices 1002, 1004, 1006 and/or 1008.

Distributed system 1000 may also include one or more data repositories 1014, 1016. These data repositories may be used to store data and other information in certain aspects. For example, one or more of the data repositories 1014, 1016 may be used to store information for techniques for disclosed herein. Data repositories 1014, 1016 may reside in a variety of locations. For example, a data repository used by server 1012 may be local to server 1012 or may be remote from server 1012 and in communication with server 1012 via a network-based or dedicated connection. Data repositories 1014, 1016 may be of different types. In certain aspects, a data repository used by server 1012 may be a database, for example, a relational database, a container database, an Exadata® storage device, or other data storage and retrieval tool such as databases provided by Oracle Corporation® and other vendors. One or more of these databases may be adapted to enable storage, update, and retrieval of data to and from the database in response to structured query language (SQL)-formatted commands.

In certain aspects, one or more of data repositories 1014, 1016 may also be used by applications to store application data. The data repositories used by applications may be of different types such as, for example, a key-value store repository, an object store repository, or a general storage repository supported by a file system.

In one embodiment, server 1012 is part of a cloud-based system environment in which various services may be offered as cloud services, for a single tenant or for multiple tenants where data, requests, and other information specific to the tenant are kept private from each tenant. In the cloud-based system environment, multiple servers may communicate with each other to perform the work requested by client devices from the same or multiple tenants. The servers communicate on a cloud-side network that is not accessible to the client devices in order to perform the requested services and keep tenant data confidential from other tenants.

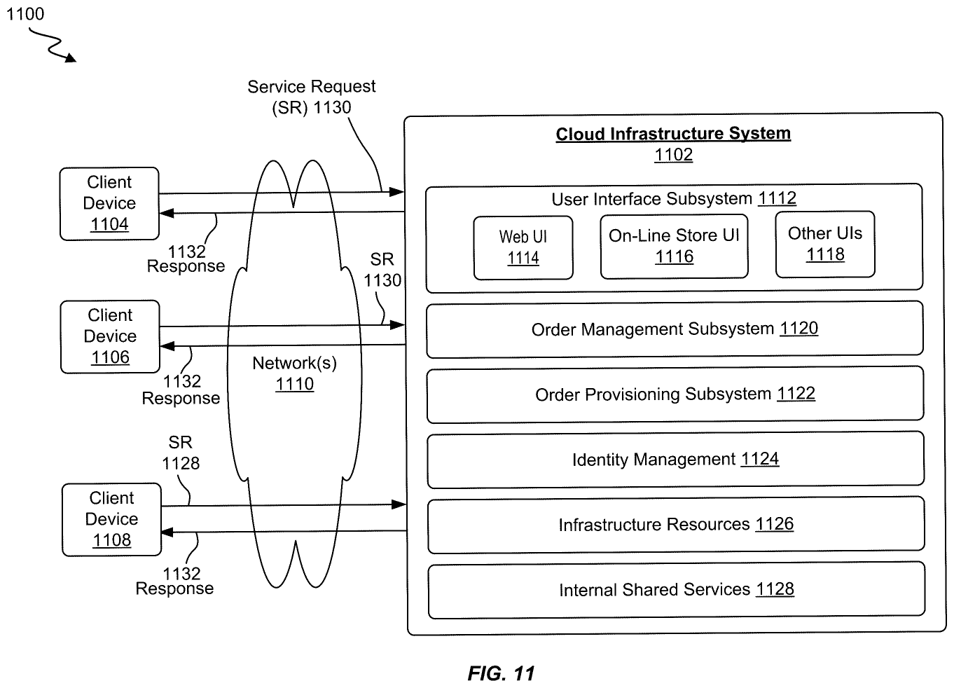

FIG. 11 is a simplified block diagram of a cloud-based system environment in accordance with some aspects of the present disclosure. In the embodiment depicted in FIG. 11, cloud infrastructure system 1102 may provide one or more cloud services that may be requested by users using one or more client computing devices 1104, 1106, and 1108. Cloud infrastructure system 1102 may comprise one or more computers and/or servers that may include those described above for server. The computers in cloud infrastructure system 1102 may be organized as general purpose computers, specialized server computers, server farms, server clusters, or any other appropriate arrangement and/or combination.

Network(s) 1110 may facilitate communication and exchange of data between clients 1104, 1106, and 1108 and cloud infrastructure system 1102. Network(s) 1110 may include one or more networks. The networks may be of the same or different types. Network(s) 1110 may support one or more communication protocols, including wired and/or wireless protocols, for facilitating the communications.

The embodiment depicted in FIG. 11 is only one example of a cloud infrastructure system and is not intended to be limiting. It should be appreciated that, in some other aspects, cloud infrastructure system 1102 may have more or fewer components than those depicted in FIG. 11, may combine two or more components, or may have a different configuration or arrangement of components. For example, although FIG. 11 depicts three client computing devices, any number of client computing devices may be supported in alternative aspects.

The term cloud service is generally used to refer to a service that is made available to users on demand and via a communication network such as the Internet by systems (e.g., cloud infrastructure system 1102) of a service provider. Typically, in a public cloud environment, servers and systems that make up the cloud service provider's system are different from the cloud customer's (“tenant's”) own on-premise servers and systems. The cloud service provider's systems are managed by the cloud service provider. Tenants can thus avail themselves of cloud services provided by a cloud service provider without having to purchase separate licenses, support, or hardware and software resources for the services. For example, a cloud service provider's system may host an application, and a user may, via a network 1110 (e.g., the Internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation®, such as database services, middleware services, application services, and others.

In certain aspects, cloud infrastructure system 1102 may provide one or more cloud services using different models such as under a Software as a Service (SaaS) model, a Platform as a Service (PaaS) model, an Infrastructure as a Service (IaaS) model, a Data as a Service (DaaS) model, and others, including hybrid service models. Cloud infrastructure system 1102 may include a suite of databases, middleware, applications, and/or other resources that enable provision of the various cloud services.

A SaaS model enables an application or software to be delivered to a tenant's client device over a communication network like the Internet, as a service, without the tenant having to buy the hardware or software for the underlying application. For example, a SaaS model may be used to provide tenants access to on-demand applications that are hosted by cloud infrastructure system 1102. Examples of SaaS services provided by Oracle Corporation® include, without limitation, various services for human resources/capital management, client relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a tenant as a cloud service to provide elastic compute and storage capabilities. Various IaaS services are provided by Oracle Corporation®.

A PaaS model is generally used to provide, as a service, platform and environment resources that enable tenants to develop, run, and manage applications and services without the tenant having to procure, build, or maintain such resources. Examples of PaaS services provided by Oracle Corporation® include, without limitation, Oracle Database Cloud Service (DBCS), Oracle Java Cloud Service (JCS), data management cloud service, various application development solutions services, and others.

A DaaS model is generally used to provide data as a service. Datasets may searched, combined, summarized, and downloaded or placed into use between applications. For example, user profile data may be updated by one application and provided to another application. As another example, summaries of user profile information generated based on a dataset may be used to enrich another dataset.

Cloud services are generally provided on an on-demand self-service basis, subscription-based, elastically scalable, reliable, highly available, and secure manner. For example, a tenant, via a subscription order, may order one or more services provided by cloud infrastructure system 1102. Cloud infrastructure system 1102 then performs processing to provide the services requested in the tenant's subscription order. Cloud infrastructure system 1102 may be configured to provide one or even multiple cloud services.

Cloud infrastructure system 1102 may provide the cloud services via different deployment models. In a public cloud model, cloud infrastructure system 1102 may be owned by a third party cloud services provider and the cloud services are offered to any general public tenant, where the tenant can be an individual or an enterprise. In certain other aspects, under a private cloud model, cloud infrastructure system 1102 may be operated within an organization (e.g., within an enterprise organization) and services provided to clients that are within the organization. For example, the clients may be various departments or employees or other individuals of departments of an enterprise such as the Human Resources department, the Payroll department, etc., or other individuals of the enterprise. In certain other aspects, under a community cloud model, the cloud infrastructure system 1102 and the services provided may be shared by several organizations in a related community. Various other models such as hybrids of the above mentioned models may also be used.

Client computing devices 1104, 1106, and 1108 may be of different types (such as devices 1002, 1004, 1006, and 1008 depicted in FIG. 10) and may be capable of operating one or more client applications. A user may use a client device to interact with cloud infrastructure system 1102, such as to request a service provided by cloud infrastructure system 1102.

In some aspects, the processing performed by cloud infrastructure system 1102 for providing chatbot services may involve big data analysis. This analysis may involve using, analyzing, and manipulating large data sets to detect and visualize various trends, behaviors, relationships, etc. within the data. This analysis may be performed by one or more processors, possibly processing the data in parallel, performing simulations using the data, and the like. For example, big data analysis may be performed by cloud infrastructure system 1102 for determining the intent of an utterance. The data used for this analysis may include structured data (e.g., data stored in a database or structured according to a structured model) and/or unstructured data (e.g., data blobs (binary large objects)).

As depicted in the embodiment in FIG. 11, cloud infrastructure system 1102 may include infrastructure resources 1130 that are utilized for facilitating the provision of various cloud services offered by cloud infrastructure system 1102. Infrastructure resources 1130 may include, for example, processing resources, storage or memory resources, networking resources, and the like.

In certain aspects, to facilitate efficient provisioning of these resources for supporting the various cloud services provided by cloud infrastructure system 1102 for different tenants, the resources may be bundled into sets of resources or resource modules (also referred to as “pods”). Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 1102 may itself internally use services 1132 that are shared by different components of cloud infrastructure system 1102 and which facilitate the provisioning of services by cloud infrastructure system 1102. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like.

Cloud infrastructure system 1102 may comprise multiple subsystems. These subsystems may be implemented in software, or hardware, or combinations thereof. As depicted in FIG. 11, the subsystems may include a user interface subsystem 1112 that enables users of cloud infrastructure system 1102 to interact with cloud infrastructure system 1102. User interface subsystem 1112 may include various different interfaces such as a web interface 1114, an online store interface 1116 where cloud services provided by cloud infrastructure system 1102 are advertised and are purchasable by a consumer, and other interfaces 1118. For example, a tenant may, using a client device, request (server 1134) one or more services provided by cloud infrastructure system 1102 using one or more of interfaces 1114, 1116, and 1118. For example, a tenant may access the online store, browse cloud services offered by cloud infrastructure system 1102, and place a subscription order for one or more services offered by cloud infrastructure system 1102 that the tenant wishes to subscribe to. The service request may include information identifying the tenant and one or more services that the tenant desires to subscribe to. For example, a tenant may place a subscription order for a chatbot related service offered by cloud infrastructure system 1102. As part of the order, the client may provide information identifying the input (e.g., utterances).

In certain aspects, such as the embodiment depicted in FIG. 11, cloud infrastructure system 1102 may comprise an order management subsystem (OMS) 1120 that is configured to process the new order. As part of this processing, OMS 1120 may be configured to: create an account for the tenant, if not done along recordeady; receive billing and/or accounting information from the tenant that is to be used for billing the tenant for providing the requested service to the tenant; verify the tenant information; upon verification, book the order for the tenant; and orchestrate various workflows to prepare the order for provisioning.

Once properly validated, OMS 1120 may then invoke the order provisioning subsystem (OPS) 1124 that is configured to provision resources for the order including processing, memory, and networking resources. The provisioning may include allocating resources for the order and configuring the resources to facilitate the service requested by the tenant order. The manner in which resources are provisioned for an order and the type of the provisioned resources may depend upon the type of cloud service that has been ordered by the tenant. For example, according to one workflow, OPS 1124 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the particular requesting tenant for providing the requested service.

Cloud infrastructure system 1102 may send a response or notification 1144 to the requesting tenant to indicate when the requested service is now ready for use. In some instances, information (e.g., a link) may be sent to the tenant that enables the tenant to start using and availing the benefits of the requested services.

Cloud infrastructure system 1102 may provide services to multiple tenants. For each tenant, cloud infrastructure system 1102 is responsible for managing information related to one or more subscription orders received from the tenant, maintaining tenant data related to the orders, and providing the requested services to the tenant or clients of the tenant. Cloud infrastructure system 1102 may also collect usage statistics regarding a tenant's use of subscribed services. For example, statistics may be collected for the amount of storage used, the amount of data transferred, the number of users, and the amount of system up time and system down time, and the like. This usage information may be used to bill the tenant. Billing may be done, for example, on a monthly cycle.

Cloud infrastructure system 1102 may provide services to multiple tenants in parallel. Cloud infrastructure system 1102 may store information for these tenants, including possibly proprietary information. In certain aspects, cloud infrastructure system 1102 comprises an identity management subsystem (IMS) 1128 that is configured to manage tenant's information and provide the separation of the managed information such that information related to one tenant is not accessible by another tenant. IMS 1128 may be configured to provide various security-related services such as identity services, such as information access management, authentication and authorization services, services for managing tenant identities and roles and related capabilities, and the like.

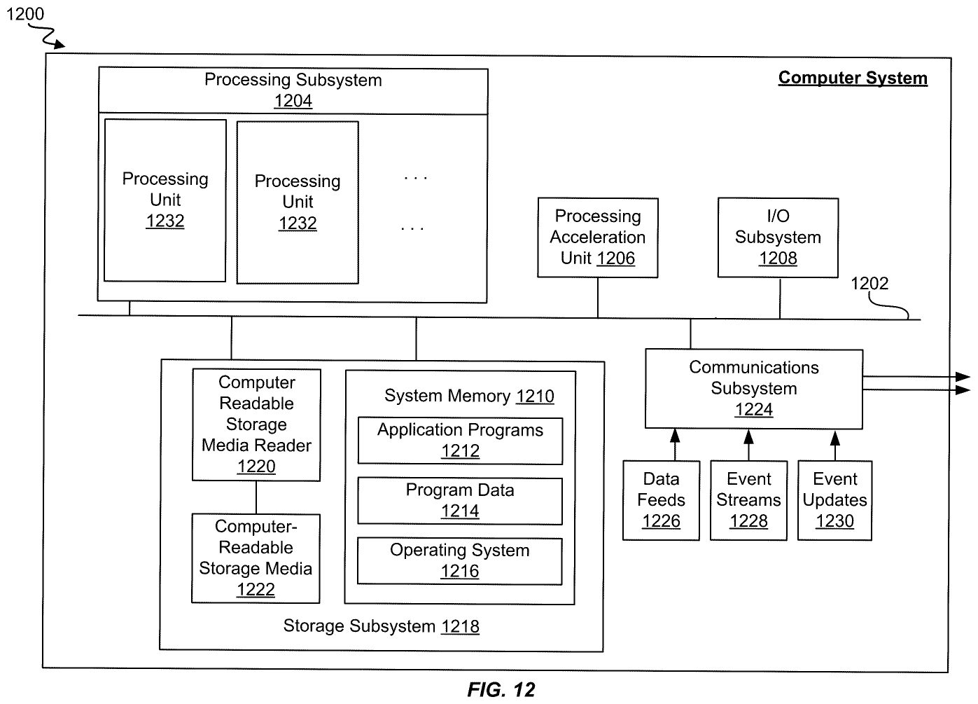

FIG. 12 illustrates an exemplary computer system 1200 that may be used to implement certain aspects of the present disclosure. As shown in FIG. 12, computer system 1200 includes various subsystems including a processing subsystem 1204 that communicates with a number of other subsystems via a bus subsystem 1202. These other subsystems may include a processing acceleration unit 1206, an I/O subsystem 1208, a storage subsystem 1218, and a communications subsystem 1224. Storage subsystem 1218 may include non-transitory computer-readable storage media including storage media 1222 and a system memory 1210.

Bus subsystem 1202 provides a mechanism for letting the various components and subsystems of computer system 1200 communicate with each other as intended. Although bus subsystem 1202 is shown schematically as a single bus, alternative aspects of the bus subsystem may utilize multiple buses. Bus subsystem 1202 may be any of several types of bus structures including a memory bus or memory controller, a peripheral bus, a local bus using any of a variety of bus architectures, and the like. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard, and the like.

Processing subsystem 1204 controls the operation of computer system 1200 and may comprise one or more processors, application specific integrated circuits (ASICs), or field programmable gate arrays (FPGAs). The processors may be single core or multicore processors. The processing resources of computer system 1200 can be organized into one or more processing units 1232, 1234, etc. A processing unit may include one or more processors, one or more cores from the same or different processors, a combination of cores and processors, or other combinations of cores and processors. In some aspects, processing subsystem 1204 can include one or more special purpose co-processors such as graphics processors, digital signal processors (DSPs), or the like. In some aspects, some, or all of the processing units of processing subsystem 1204 can be implemented using customized circuits, such as application specific integrated circuits (ASICs), or field programmable gate arrays (FPGAs).

In some aspects, the processing units in processing subsystem 1204 can execute instructions stored in system memory 1210 or on computer readable storage media 1222. In various aspects, the processing units can execute a variety of programs or code instructions and can maintain multiple concurrently executing programs or processes. At any given time, some, or all of the program code to be executed can be resident in system memory 1210 and/or on computer-readable storage media 1222 including potentially on one or more storage devices. Through suitable programming, processing subsystem 1204 can provide various functionalities described above. In instances where computer system 1200 is executing one or more virtual machines, one or more processing units may be allocated to each virtual machine.

In certain aspects, a processing acceleration unit 1206 may optionally be provided for performing customized processing or for off-loading some of the processing performed by processing subsystem 1204 so as to accelerate the overall processing performed by computer system 1200.

I/O subsystem 1208 may include devices and mechanisms for inputting information to computer system 1200 and/or for outputting information from or via computer system 1200. In general, use of the term input device is intended to include all possible types of devices and mechanisms for inputting information to computer system 1200. User interface input devices may include, for example, a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices such as the Meta Quest® controller, Microsoft Kinect® motion sensor, the Microsoft Xbox® 360 game controller, or devices that provide an interface for receiving input using gestures and spoken commands. User interface input devices may also include eye gesture recognition devices such as a blink detector that detects eye activity (e.g., “blinking” while taking pictures and/or making a menu selection) from users and transforms the eye gestures as inputs to an input device. Additionally, user interface input devices may include voice recognition sensing devices that enable users to interact with voice recognition systems (e.g., Siri® navigator or Amazon Alexa®) through voice commands.

Other examples of user interface input devices include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, QR code readers, barcode readers, 3D scanners, 3D printers, laser rangefinders, and eye gaze tracking devices. Additionally, user interface input devices may include, for example, medical imaging input devices such as computed tomography, magnetic resonance imaging, position emission tomography, and medical ultrasonography devices. User interface input devices may also include, for example, audio input devices such as MIDI keyboards, digital musical instruments, and the like.

In general, use of the term output device is intended to include all possible types of devices and mechanisms for outputting information from computer system 1200 to a user or other computer. User interface output devices may include a display subsystem, indicator lights, or non-visual displays such as audio output devices, etc. The display subsystem may be any device for outputting a digital picture. Example display devices include flat panel display devices such as those using a light emitting diode (LED) display, a liquid crystal display (LCD) or plasma display, a projection device, a touch screen, a desktop or laptop computer monitor, and the like. As another example, wearable display devices such as Meta Quest® or Microsoft HoloLens® may be mounted to the user for displaying information. User interface output devices may include, without limitation, a variety of display devices that visually convey text, graphics, and audio/video information such as monitors, printers, speakers, headphones, automotive navigation systems, plotters, voice output devices, and modems.

Storage subsystem 1218 provides a repository or data store for storing information and data that is used by computer system 1200. Storage subsystem 1218 provides a tangible non-transitory computer-readable storage medium for storing the basic programming and data constructs that provide the functionality of some aspects. Storage subsystem 1218 may store software (e.g., programs, code modules, instructions) that when executed by processing subsystem 1204 provides the functionality described above. The software may be executed by one or more processing units of processing subsystem 1204. Storage subsystem 1218 may also provide a repository for storing data used in accordance with the teachings of this disclosure.

Storage subsystem 1218 may include one or more non-transitory memory devices, including volatile and non-volatile memory devices. As shown in FIG. 12, storage subsystem 1218 includes a system memory 1210 and a computer-readable storage media 1222. System memory 1210 may include a number of memories including a volatile main random access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system 1200, such as during start-up, may typically be stored in the ROM. The RAM typically contains data and/or program modules that are presently being operated and executed by processing subsystem 1204. In some implementations, system memory 1210 may include multiple different types of memory, such as static random access memory (SRAM), dynamic random access memory (DRAM), and the like.

By way of example, and not limitation, as depicted in FIG. 12, system memory 1210 may load application programs 1212 that are being executed, which may include various applications such as Web browsers, mid-tier applications, relational database management systems (RDBMS), etc., program data 1214, and an operating system 1216. By way of example, operating system 1216 may include various versions of Microsoft Windows®, Apple Macintosh®, and/or Linux® operating systems, a variety of commercially-available UNIX® or UNIX-like operating systems (including without limitation the variety of GNU/Linux operating systems, the Oracle Linux®, Google Chrome® OS, and the like) and/or mobile operating systems such as iOS, Windows® Phone, Android® OS, and others.

Computer-readable storage media 1222 may store programming and data constructs that provide the functionality of some aspects. Computer-readable media 1222 may provide storage of computer-readable instructions, data structures, program modules, and other data for computer system 1200. Software (programs, code modules, instructions) that, when executed by processing subsystem 1204 provides the functionality described above, may be stored in storage subsystem 1218. By way of example, computer-readable storage media 1222 may include non-volatile memory such as a hard disk drive, a magnetic disk drive, an optical disk drive such as a CD ROM, digital video disc (DVD), a Blu-Ray® disk, or other optical media. Computer-readable storage media 1222 may include, but is not limited to, Zip® drives, flash memory cards, universal serial bus (USB) flash drives, secure digital (SD) cards, DVD disks, digital video tape, and the like. Computer-readable storage media 1222 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, dynamic random access memory (DRAM)-based SSDs, magnetoresistive RAM (MRAM) SSDs, and hybrid SSDs that use a combination of DRAM and flash memory based SSDs.

In certain aspects, storage subsystem 1218 may also include a computer-readable storage media reader 1220 that can further be connected to computer-readable storage media 1222. Reader 1220 may receive and be configured to read data from a memory device such as a disk, a flash drive, etc.

In certain aspects, computer system 1200 may support virtualization technologies, including but not limited to virtualization of processing and memory resources. For example, computer system 1200 may provide support for executing one or more virtual machines. In certain aspects, computer system 1200 may execute a program such as a hypervisor that facilitated the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 1200. Accordingly, multiple operating systems may potentially be run concurrently by computer system 1200.

Communications subsystem 1224 provides an interface to other computer systems and networks. Communications subsystem 1224 serves as an interface for receiving data from and transmitting data to other systems from computer system 1200. For example, communications subsystem 1224 may enable computer system 1200 to establish a communication channel to one or more client devices via the Internet for receiving and sending information from and to the client devices. For example, the communications subsystem may be used to transmit a response to a user regarding the inquiry for a chatbot.

Communications subsystem 1224 may support both wired and/or wireless communication protocols. For example, in certain aspects, communications subsystem 1224 may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 802.XX family standards, or other mobile communication technologies, or any combination thereof), global positioning system (GPS) receiver components, and/or other components. In some aspects communications subsystem 1224 can provide wired network connectivity (e.g., Ethernet) in addition to or instead of a wireless interface.

Communications subsystem 1224 can receive and transmit data in various forms. For example, in some aspects, in addition to other forms, communications subsystem 1224 may receive input communications in the form of structured and/or unstructured data feeds 1226, event streams 1228, event updates 1230, and the like. For example, communications subsystem 1224 may be configured to receive (or send) data feeds 1226 in real-time from users of social media networks and/or other communication services such as Twitter® feeds, Facebook® updates, web feeds such as Rich Site Summary (RSS) feeds, and/or real-time updates from one or more third party information sources.

In certain aspects, communications subsystem 1224 may be configured to receive data in the form of continuous data streams, which may include event streams 1228 of real-time events and/or event updates 1230, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1224 may also be configured to communicate data from computer system 1200 to other computer systems or networks. The data may be communicated in various different forms such as structured and/or unstructured data feeds 1226, event streams 1228, event updates 1230, and the like to one or more databases that may be in communication with one or more streaming data source computers coupled to computer system 1200.

Computer system 1200 can be one of various types, including a handheld portable device (e.g., an iPhone® cellular phone, an iPad® computing tablet, a personal digital assistant (PDA)), a wearable device (e.g., a Meta Quest® head mounted display), a personal computer, a workstation, a mainframe, a kiosk, a server rack, or any other data processing system. Due to the ever-changing nature of computers and networks, the description of computer system 1200 depicted in FIG. 12 is intended only as a specific example. Many other configurations having more or fewer components than the system depicted in FIG. 12 are possible. Based on the disclosure and teachings provided herein, a person of ordinary skill in the art can appreciate other ways and/or methods to implement the various aspects.

Although specific aspects have been described, various modifications, alterations, alternative constructions, and equivalents are possible. Embodiments are not restricted to operation within certain specific data processing environments, but are free to operate within a plurality of data processing environments. Additionally, although certain aspects have been described using a particular series of transactions and s, it should be apparent to those skilled in the art that this is not intended to be limiting. Although some flowcharts describe operations as a sequential process, many of the operations can be performed in parallel or concurrently. In addition, the order of the operations may be rearranged. A process may have additional s not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claimed is:

1. A computer-implemented method comprising:
receiving a request comprising identifiers corresponding to a subject via an interface associated with a user;
accessing, in response to the request, one or more unstructured portions corresponding to the subject via an application programmable interface (API) from an electronic health record, wherein the one or more unstructured portions are in binary format;
filtering, via the API, the one or more unstructured portions into filtered data based on a set of filtering criteria;
transforming the filtered data into a long record data that is semi-structured;
processing the long record data into a text-based format;
transforming the text-based format into a structured output in a natural language format using one or more generative artificial intelligence (GenAI) models, wherein the transformation includes:
generating a prompt in the natural language format based on the text-based format;
extracting, in response to the prompt, one or more data elements from the text-based format; and
processing the one or more data elements into the structured output based on a set of transformation criteria that defines a format of the structured output; and
outputting a result corresponding to the structured output via the interface associated with the user.

2. The computer-implemented method of claim 1, further comprising:
assigning user scores to the one or more data elements or the structured output displayed on the interface, wherein the user scores represent a review or validation of an accuracy of the one or more data elements or the structured output by the user.

3. The computer-implemented method of claim 1, further comprising:
accessing, via an annotation tool integrated within the interface, a set of annotations defined by the user based on the text-based format, wherein an annotation of the set of annotations represents an assigned label to a designated data element present within the format and a location of the designated data element associated with the assigned label;
comparing the set of annotations with the text-based format to identify the text-based format that aligns with the set of annotations;
enriching the text-based format with the set of annotations; and
generating, based on the enrichment, annotated data that is used as a baseline dataset by an auto-evaluator to assign evaluation scores to the one or more data elements or the structured output.

4. The computer-implemented method of claim 3, wherein the evaluation scores generated via the auto-evaluator of at least the one or more GenAI models are used to train the other one or more GenAI models including:
comparing the evaluation scores with user scores associated with the one or more data elements or the structured output;
iteratively improving the auto-evaluator to align the evaluation scores with the user scores based on a threshold; and
establishing a confidence threshold for the auto-evaluator based on the iterative improvement.

5. The computer-implemented method of claim 1, wherein the one or more data elements comprise visit rationales or follow-up instructions.

6. The computer-implemented method of claim 1, wherein the set of transformation criteria that defines the format of the structured output includes format requirements and logic requirements.

7. The computer-implemented method of claim 1, wherein the structured output comprises a summary including one or more of: subject profile (e.g., name, age, active conditions, allergies, care team), care team, clinical summary associated with encounters (e.g., medical history, diagnoses, treatments, lab results, medications), social factors (e.g., access to transportation, money and resources, family and home situation), future appointments or a combination thereof.

8. The computer-implemented method of claim 7, wherein the structured output further includes supporting facts that provide additional details to reinforce and substantiate reliability of the summary.

9. The computer-implemented method of claim 1, wherein the result further comprises talking points, call activity logs (e.g., calls made, call summaries, interactions with the subject), care plan updates (e.g., modifications to treatment plans, setting of goals) or reminders (e.g., scheduled tasks, follow-ups for care managers).

10. A system comprising:
one or more data processors; and
the one or more non-transitory computer readable storage medium containing instructions which, when executed on the one or more data processors, cause the one or more data processors to perform operations including:
receiving a request comprising identifiers corresponding to a subject via an interface associated with a user;
accessing, in response to the request, one or more unstructured portions corresponding to the subject via an application programmable interface (API) from an electronic health record, wherein the one or more unstructured portions are in binary format;
filtering, via the API, the one or more unstructured portions into filtered data based on a set of filtering criteria;
transforming the filtered data into a long record data that is semi-structured;
processing the long record data into a text-based format;
transforming the text-based format into a structured output in a natural language format using one or more generative artificial intelligence (GenAI) models, wherein the transformation includes:
generating a prompt in the natural language format based on the text-based format;
extracting, in response to the prompt, one or more data elements from the text-based format; and
processing the one or more data elements into the structured output based on a set of transformation criteria that defines a format of the structured output; and
outputting a result corresponding to the structured output via the interface associated with the user.

11. The system of claim 10, further comprising:
assigning user scores to the one or more data elements or the structured output displayed on the interface, wherein the user scores represent a review or validation of an accuracy of the one or more data elements or the structured output by the user.

12. The system of claim 10, further comprising:
accessing, via an annotation tool integrated within the interface, a set of annotations defined by the user based on the text-based format, wherein an annotation of the set of annotations represents an assigned label to a designated data element present within the format and a location of the designated data element associated with the assigned label;
comparing the set of annotations with the text-based format to identify the text-based format that aligns with the set of annotations;
enriching the text-based format with the set of annotations; and
generating, based on the enrichment, annotated data that is used as a baseline dataset by an auto-evaluator to assign evaluation scores to the one or more data elements or the structured output.

13. The system of claim 12, wherein the evaluation scores generated via the auto-evaluator of at least the one or more GenAI models are used to train the other one or more GenAI models including:
comparing the evaluation scores with user scores associated with the one or more data elements or the structured output;
iteratively improving the auto-evaluator to align the evaluation scores with the user scores based on a threshold; and
establishing a confidence threshold for the auto-evaluator based on the iterative improvement.

14. The system of claim 10, wherein the structured output comprises a summary including one or more of: subject profile (e.g., name, age, active conditions, allergies, care team), care team, clinical summary associated with encounters (e.g., medical history, diagnoses, treatments, lab results, medications), social factors (e.g., access to transportation, money and resources, family and home situation), future appointments or a combination thereof.

15. The system of claim 14, wherein the structured output further includes supporting facts that provide additional details to reinforce and substantiate reliability of the summary.

16. A computer-program product tangibly embodied in a non-transitory machine-readable storage medium, including instructions configured to cause one or more data processors to perform operations including:
receiving a request comprising identifiers corresponding to a subject via an interface associated with a user;
accessing, in response to the request, one or more unstructured portions corresponding to the subject via an application programmable interface (API) from an electronic health record, wherein the one or more unstructured portions are in binary format;
filtering, via the API, the one or more unstructured portions into filtered data based on a set of filtering criteria;
transforming the filtered data into a long record data that is semi-structured;
processing the long record data into a text-based format;
transforming the text-based format into a structured output in a natural language format using one or more generative artificial intelligence (GenAI) models, wherein the transformation includes:
generating a prompt in the natural language format based on the text-based format;
extracting, in response to the prompt, one or more data elements from the text-based format; and
processing the one or more data elements into the structured output based on a set of transformation criteria that defines a format of the structured output; and
outputting a result corresponding to the structured output via the interface associated with the user.

17. The computer-program product of claim 16, further comprising:
assigning user scores to the one or more data elements or the structured output displayed on the interface, wherein the user scores represent a review or validation of an accuracy of the one or more data elements or the structured output by the user.

18. The computer-program product of claim 16, further comprising:
accessing, via an annotation tool integrated within the interface, a set of annotations defined by the user based on the text-based format, wherein an annotation of the set of annotations represents an assigned label to a designated data element present within the format and a location of the designated data element associated with the assigned label;
comparing the set of annotations with the text-based format to identify the text-based format that aligns with the set of annotations;
enriching the text-based format with the set of annotations; and
generating, based on the enrichment, annotated data that is used as a baseline dataset by an auto-evaluator to assign evaluation scores to the one or more data elements or the structured output.

19. The computer-program product of claim 18, wherein the evaluation scores generated via the auto-evaluator of at least the one or more GenAI models are used to train the other one or more GenAI models including:
comparing the evaluation scores with user scores associated with the one or more data elements or the structured output;
iteratively improving the auto-evaluator to align the evaluation scores with the user scores based on a threshold; and
establishing a confidence threshold for the auto-evaluator based on the iterative improvement.

20. The computer-program product of claim 16, wherein the structured output comprises a summary including one or more of: subject profile (e.g., name, age, active conditions, allergies, care team), care team, clinical summary associated with encounters (e.g., medical history, diagnoses, treatments, lab results, medications), social factors (e.g., access to transportation, money and resources, family and home situation), future appointments or a combination thereof.
